SEQUENCE LISTING (13 sequences)

SEQ ID NO: 1 - protein, 8 aa, synthetic construct
NTSGMGVG

SEQ ID NO: 2 - protein, 16 aa, synthetic construct
HIWWDDVKRYNPALKS

SEQ ID NO: 3 - protein, 8 aa, synthetic construct
TLPHFFDY

SEQ ID NO: 4 - protein, 118 aa, synthetic construct
QVTLKVSGPGILQPSQTLSLTCSFSGFSLNTSGMGVGWIRQPSGKGLEWLAHIWWDDVKRYNPALKSRLTISKDTSGSQVFLKIASVDTADTATYYCARTLPHFFDYWGQGTTLTVSS

SEQ ID NO: 5 - protein, 10 aa, synthetic construct
RASSSVSYMY

SEQ ID NO: 6 - protein, 7 aa, synthetic construct
RTSNLAS

SEQ ID NO: 7 - protein, 11 aa, synthetic construct
KQYHSYPPTWT

SEQ ID NO: 8 - protein, 109 aa, synthetic construct
DIQMTQSPAIMSASPGEKVTISCRASSSVSYMYWYQQKSGSSPKPWIYRTSNLASGVPARFSGSGSGTSYSLTISSMEAEDAATYYCKQYHSYPPTWTFGGGTKLEIKR

SEQ ID NO: 9 - protein, 8 aa, synthetic construct
STSGMGVG

SEQ ID NO: 10 - protein, 16 aa, synthetic construct
HIWWDDVKRYNPSLKS

SEQ ID NO: 11 - protein, 8 aa, synthetic construct
TLPHFHDY

SEQ ID NO: 12 - protein, 118 aa, synthetic construct
QVTLKESGPTLVKPTQTLTLTCTFSGFSLSTSGMGVGWIRQPPGKALEWLAHIWWDDVKRYNPSLKSRLTITKDTSGSQVVLTMTNMDPVDTATYYCARTLPHFHDYWGQGTTLTVSS

SEQ ID NO: 13 - protein, 109 aa, synthetic construct
DIQMTQSPSTLSASVGDRVTITCRASSSVSYMYWYQQKPGKAPKPLIYRTSNLASGVPSRFSGSGSGTEYTLTISSLQPDDFATYYCKQYHSYPPTWTFGGGTKVEIKR

(19) United States
(12) Patent Application Publication
(10) Pub. No.: US 2025/0170238 A1
(43) Pub. Date: May 29, 2025

(54) ANTI-ALARMIN BINDING MOLECULES AND TREATMENT OF PNEUMONITIS

(71) Applicants: Daniel WHITE, Suwanee, GA (US); Crystal JACKSON, Suwanee, GA (US); Petros NIKOLINOKOS, Suwanee, GA (US); Sven SWANSON, Suwanee, GA (US); Lanier Biotherapeutics, Inc., Suwanee, GA (US)

(72) Inventors: Daniel WHITE, Suwanee, GA (US); Crystal JACKSON, Jefferson, GA (US); Petros NIKOLINOKOS, Suwanee, GA (US); Sven SWANSON, Suwanee, GA (US)

(21) Appl. No.: 18/841,520

(22) PCT Filed: Feb. 24, 2023
(86) PCT No.: PCT/US2023/063267

§ 371 Date: Aug. 26, 2024

Related U.S. Application Data
(60) Provisional application No. 63/313,861, filed on Feb. 25, 2022.
(60) Provisional application No. 63/380,506, filed on Oct. 21, 2022.
(60) Provisional application No. 63/479,623, filed on Jan. 12, 2023.

(51) Int. Cl. A61K 39/395; A61P 11/00

(52) U.S. Cl. CPC A61K 39/3955; A61P 11/00

(57) ABSTRACT

The present disclosure provides methods and compositions for the treatment of pneumonitis (including checkpoint inhibitor induced pneumonitis), fibrosis, and type 2 inflammatory disorders comprising administering anti-alarmin binding molecules to a subject in need thereof. In son embodiments treatment comprises administering one or more checkpoint inhibitors conjugated to a therapeutic moiety comprising a cytotoxic agent, a therapeutic agent, a radioisotope, an ultrasound sensitizer, or an exosome secretion inhibitor.

### CROSS REFERENCE

The present application claims priority to and benefit from U.S. Provisional Application No. 63/313,861 filed Feb. 25, 2022, U.S. Provisional Application No. 63/380,506 filed on Oct. 21, 2022, and U.S. Provisional Application No. 63/479,623 filed on Jan. 12, 2023, the entire contents of each of which are herein incorporated by reference

### SEQUENCE LISTING

The contents of the electronic sequence listing (LNER_008_04WO_SeqList_ST26.xml; Size: 12,756 bytes; and Date of Creation: Feb. 21, 2023) are herein incorporated by reference in its entirety.

### FIELD OF THE INVENTION

The present disclosure relates generally to alarmin antagonists and methods of using the foregoing.

### BACKGROUND

Type 2 inflammation is a specific type of immune response that can have host-protective activity, for example mediating protective immunity to parasitic helminth infection, but dysregulation can lead to pathogenesis. Overexpression of type 2 cytokines and hyperreactive type 2 immune responses can lead to the development of many allergic and fibrotic diseases such as atopic dermatitis (eczema), chronic rhinosinusitis, allergic rhinitis, eosinophilic esophagitis, fibrosis, asthma and anaphylaxis. The immune system has different strategies to deal with different pathogens. The mechanisms utilized to identify and kill microorganisms such as bacteria or viruses is not to the same as those employed to eliminate parasite worms. Therefore, the immune system has specialized components that eliminate and prevent invasions and infections in different ways. Type 2 immune activation is one pattern of activation whereinT cells become activated by other types of immune cells to recognize specific pathogens. In type 2 immunity, some cells undergo further changes to become what are called T helper cells (“Th” cells). T helper cells play a key role in coordinating the immune response through releasing specific immune-signaling molecules, called cytokines. Cytokines influence the activity of a variety of other cells in the immune system to act in specific ways. Based on the signaling and activation from other immune cells, T helper cells generate one of two overarching types of immune responses. Overall, a Th type response (or type 1 inflammatory response) is better at producing an immune response that is effective at targeting viruses and bacteria. In contrast, a Th2 type response is better at eliminating certain parasites, like tapeworms or nematodes. During a type 2 inflammatory response (Th2 response). T helper cells release cytokines such as IL-4, IL-5, IL-9, and IL-13. The Th2 response also promotes the formation of a specific type of antibody, termed IgE antibodies. Specific immune cells, including mast cells, basophils, and eosinophils are activated to secrete mucus, promote swelling, contract smooth muscle cells, and release particles that destroy parasites. During an active infection, these responses help rid the body of the invading parasite. A Th2 type immune response can be very helpful in fighting infections, however, dysregulation, hyperreactivity, and chronic activation can drive the onset of disease. In such cases, too many T cells may become activated by the Th2 signaling pathway, some of which may differentiate into memory cells that confer long-lived immunity and cause long-term changes in the immune response. This can lead to chronic inflammation, triggered by recurrent antigen presentations. When the Th2 pathway is dysregulated in this fashion, it is more often referred to as type 2 inflammation. This inflammatory pathway is, at times, activated by non-infectious stimuli. When the immune system is over-sensitized, severe type 2 responses to external stimuli like pollen, animal dander, dust, or certain foods can lead to inflammation triggering an allergic response caused by hyperactivation of the Th2 pathway. Type 2 inflammation is clearly implicated in atopic diseases are exacerbated by certain environmental allergic triggers. Atopic diseases are closely related and individuals presenting with one disease are predisposed to additional exacerbations more so than someone in the general population. Some of the diseases of this type include: Atopic dermatitis (commonly called eczema); Chronic rhinosinusitis (sometimes with nasal polyps; CRSwNP), Asthma, Chronic spontaneous urticaria. The type 2 inflammatory pathway is also involved in life-threatening anaphylactic allergic reactions. For example, some people have such reactions to peanuts, bee stings, or other triggers. Exaggerated type 2 inflammation may also be playing a role in some autoimmune diseases, such as multiple sclerosis. Researchers have been studying the type 2 inflammatory pathway in these diseases, and exaggerated type 2 inflammation may be an underlying cause.

Type 2 immunity exhibits many host-protective functions, including maintenance of metabolic homeostasis, suppression of excessive type 1 inflammation, maintenance of barrier defense and regulation of tissue regeneration. However, excessive and chronic activation of the type 2 response can lead to allergic disease, reduction in pathogen defense and lethal fibrosis. Type 2 responses can be initiated and maintained through diverse mechanisms, including, among others, classical antigen presentation and recognition: epithelial chemo-sensing and the innate lymphoid axis; epithelial damage, alarmin release and platelet-derived factors. Type 2 cytokines orchestrate tissue repair and fibrosis directly and indirectly by targeting a wide array of immune and non-immune cell types, including macrophages, fibroblasts, epithelial cells and endothelial cells. Therapeutics that block type 2-driven fibrosis must avoid reducing the critical tissue-regenerative functions of IL-4 and IL-13. These cytokines have been shown to directly facilitate tissue repair by activating tissue progenitor cell populations and by targeting the proliferation of various epithelial cell populations. (Gieseck, et al. Nat Rev Immunol 18, 62-76 (2018))

Immune checkpoint inhibitors (ICIs), such as anti-PD1 and anti-CTLA4 antibodies, are important therapeutic agents used in advanced malignancies such as melanoma and non-small cell lung carcinoma. However, a wide array of immune related adverse effects can result from ICI therapy, with one of the most serious (and potentially lethal) being checkpoint inhibitor pneumonitis (CIP). A clinical conundrum can occur where the treatment is worse than the disease—for example, patients can die from the CIP although the tumor has been treated successfully. Thus, patients having a positive genotype to PD1, PD-L1, or CTLA4 who receive checkpoint inhibitor therapy may be good candidates for cancer management and inflammation suppression.

CIP is an immune-related adverse event (irAE), potentially resulting in significant morbidity with possible discontinuation of therapy and possible mortality. Overall, the incidence of CIP is estimated to be between 3% and 6%.(The risk ratio of some irAEs of clinical interest 24.01% for pneumonitis (Wang et al, JAMA Oncol. 2019; 5(7):1008-1019). Higher rates of pneumonitis have been observed in non-small cell lung cancer and renal cell carcinoma versus those of melanoma. In patients with non-small cell lung carcinoma, the incidence and severity of pneumonitis has been shown to be higher in patients undergoing treatment with PD-1 inhibitors compared with those undergoing treatment with PD-L1 inhibitors (3.6% vs. 1.3%, respectively), with a lower incidence in those patients undergoing treatment with CTLA-4 inhibitors (Rakshit et al., Chest 2017). Pneumonitis is more likely to manifest in patients receiving ICI combination therapy compared with those receiving monotherapy (Naido et al., J Clin Oncol 2017). The time to pneumonitis onset is widely variable, reported to range from 9 days to over 19 months after initiation of therapy, with a median time of onset of 2.8 months. Onset has been shown to occur earlier in patients with lung cancer compared with those with melanoma (2.1 versus 5.2 months, respectively) (Delaunay et al, Eur Respir J 2019).

Once a patient moves from a cough to stage 2, then typically they would receive conventional therapies for treatment of CIP include high-dose corticosteroids such as prednisone. Corticosteroid treatment used to treat CIP can have several side effects. These may include increased risk of infection, high blood sugar, high blood pressure, weight gain, mood changes, and bone loss. In addition to corticosteroids, other immunosuppressive therapies may be used in the treatment of checkpoint inhibitor pneumonitis. These may include drugs such as infliximab, mycophenolate mofetil, or cyclophosphamide. However, the use of immunosuppressive medications can increase the risk of infection and other complications.

Blocking immune checkpoints can enhance the type 2 immune response, as these immune checkpoints are known to suppress T cell activation and cytokine production, which are key components of the type 2 immune response. The type 2 immune response is a type of immune response that is involved in the defense against certain types of pathogens, such as parasitic worms and certain viruses. It is characterized by the activation of specialized immune cells, such as T helper 2 (Th2) cells, and the production of specific cytokines, such as interleukin-4 (IL-4), IL-5, and IL-13. Type 2 cytokines can cause various side effects, depending on the specific cytokine and the context in which it is produced. Some possible side effects include allergic reactions, tissue inflammation, and suppression of the immune response. Current approaches targeting these cytokines, such as monoclonal antibodies that target IL-4, IL-5, and IL-13, yield mixed clinical results and it is difficult to target all cytokines simultaneously.

Several alarmins have been implicated in the induction of type 2 inflammation, including IL-25, IL-33 and Thymic stromal lymphopoietin (TSLP). IL-25 is produced by epithelia cells and can promote the differentiation of Th2 cells and the production of type 2 cytokines, such as IL-5 and IL-13. IL-25 has been shown to be involved in the regulation of allergic responses and the defense against parasitic infections. IL-33 is produced by epithelial and stromal cells and can activate multiple immune cell types, including eosinophils and Th2 cells. IL-33 has been implicated in the pathogenesis of allergic diseases and asthma. TSLP is produced by epithelial cells and can activate dendritic cells and promote the differentiation of Th2 cells. TSLP has been implicated in the regulation of allergic responses and the pathogenesis of atopic dermatitis. By blocking the activity of these alarmins, it may be possible to reduce inflammation and tissue damage in a variety of conditions.

However, the development of anti-alarmin therapy presents several challenges and difficulties, including identifying the specific alarmins that are involved in disease, ensuring the safety and efficacy of these therapies, and overcoming technical and regulatory hurdles.

Therefore there remains an urgent need to develop effective and safe anti-alarmin therapeutics to prevent CIP or other inflammatory diseases.

### SUMMARY

Present disclosure provides compositions and methods for preventing or treating pneumonitis. In some embodiments, the present disclosure provides methods of preventing or treating pneumonitis in a subject in need thereof comprising administering an anti-alarmin binding molecule to the subject. In some embodiments, the pneumonitis is the result of interstitial lung disease, viral infection, autoimmune disease, allergy, inhalation of occupational debris, dusts, fibers, fumes or vapors, inhalation of chemicals or molds, sepsis, adverse reaction to medications, aspirin overdose, hypersensitivity to environmental antigens, overexposure to chlorine, exposure to herbicides, fluorocarbons, radiation, chemotherapy, and/or treatment with one or more immune checkpoint inhibitors. In some embodiments, the anti-alarmin binding molecule is administered after or prior to the onset of pneumonitis symptoms.

Present disclosure also provides methods of preventing or treating checkpoint inhibitor-induced pneumonitis (CIP) in a subject in need thereof comprising administering an neutralizing anti-alarmin binding molecule to the subject. In some embodiments, the checkpoint inhibitor is selected from an anti-programmed death receptor-1(PD1) molecule, an anti-programmed death ligand 1 (PD-L1) molecule, an anti-cytotoxic T-lymphocyte associated protein 4 (CTLA4) molecule, an anti-Lymphocyte-activation gene 3 (LAG3) molecule, an anti-T-cell immunoreceptor with Ig and ITIM domains (TIGIT) molecule, an anti-T-cell immunoglobulin and mucin domain-containing protein 3 (TIM-3) molecule, an anti-V-domain Ig suppressor of T cell activation (VISTA) molecule, an anti-B and T lymphocyte attenuator (BTLA) molecule, an anti-Sialic acid-binding Ig-like lectin 15 (Siglec-15) molecule, and an anti-CD96 molecule. In some embodiments, the anti-PD1 molecule is an anti-PD1 antibody selected from a group consisting of pembrolizumab, nivolumab, cemiplimab, dostarlimab, spartalizumab, camrelizumab, sintilimab, tislelizumab, toripalimab, and retifanlimab. In some embodiments, the anti-CTLA4 molecule is an anti-CTLA4 antibody selected from a group consisting of ipilimumab, tremelimumab, BMS-986249, quavonlimab, and AGEN1884. In some embodiments, the anti-PD-L1 molecule is an anti-PD-L1 antibody selected from atezolizumab, avelumab, and durvalumab. In some embodiments, the treatment with one or more checkpoint inhibitors comprises treatment with two checkpoint inhibitors. In certain embodiments, the two checkpoint inhibitors comprise an anti-PD1 antibody and an anti-CTLA4 antibody. In some embodiments, the treatment with one or more checkpoint inhibitors comprises a cell therapy comprising CAT-T cells or allogeneic T cells expressing one or more checkpoint inhibitors. In some embodiments, the treatment with one or more checkpoint inhibitors comprises a gene therapy comprising viral vectors expressing one or more checkpoint inhibitors. In some embodiments, the treatment with one or more checkpoint inhibitors comprises one or more checkpoint inhibitors conjugated to a therapeutic moiety comprising a cytotoxic agent, a therapeutic agent, a radioisotope, an ultrasound sensitizer, or an exosome secretion inhibitor. In some embodiments, the cytotoxic agent comprises taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, the therapeutic agent comprises antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). In some embodiments, the radioisotope is radioactive iodine. In some embodiments, the ultrasound sensitizer comprises porphyrins, porphyrin isomers and expanded porphyrins. In some embodiments, the exosome secretion inhibitor comprises Manumycin A, GW4869, cannabidiol and endothelin receptor antagonists.

In some embodiments, the subject has previously received treatment with one or more checkpoint inhibitors. In some embodiments, the anti-alarmin binding molecule is administered at least 1, 2, 3, 4, 5, 6, 7, 8, 9, or 10 days after the administration of one or more checkpoint inhibitors. In some embodiments, the anti-alarmin binding molecule is administered concurrently with or prior to one or more checkpoint inhibitors. In certain embodiments, the anti-alarmin binding molecule is administered at least 1, 2, 3, 4, 5, 6, 7, 8, 9, or 10 days prior to the administration of one or more checkpoint inhibitors.

Present disclosure also provides methods of preventing or treating fibrosis in a subject in need thereof comprising administering an anti-alarmin binding molecule to the subject. In some embodiments, the fibrosis is pulmonary fibrosis, liver fibrosis, cardiac fibrosis, renal fibrosis, skin fibrosis, gastrointestinal fibrosis, colon fibrosis, or pancreatic fibrosis. In one embodiment, the fibrosis is idiopathic pulmonary fibrosis or bleomycin-induced fibrosis. In some embodiments, the fibrosis is the result of interstitial lung disease, viral infection, autoimmune disease, allergy, inhalation of occupational debris, dusts, fibers, fumes or vapors, inhalation of chemicals or molds, sepsis, adverse reaction to medications, aspirin overdose, hypersensitivity to environmental antigens, overexposure to chlorine, exposure to herbicides, fluorocarbons, radiation, chemotherapy, immune dysregulation, and/or treatment with one or more immune checkpoint inhibitors. In some embodiments, the treatment for fibrosis results in a reduction in the ratio of lymphocytes relative to the total bronchoalveolar cells, a reduction in the ratio of neutrophils relative to the total bronchoalveolar cell, a reduction in total collagen, a reduction in fibronectin, and/or a reduction in one or more molecular pro-fibrotic mediators. In some embodiments, the molecular pro-fibrotic mediator is selected from transforming growth factor beta (TGF-β), connective tissue growth factor (CTGF), platelet-derived growth factor (PDGF), fibroblast growth factor (FGF), endothelin-1 (ET-1), IL-4, IL-5, IL-13, IL-21, MCP-1, MIP-1β, angiogenic factors (VEGF), TNF-α, peroxisome proliferator-activated receptors (PPARs), acute phase proteins (SAP), caspases, Angiotensin II, and endothelin (ET).

Present disclosure further provides methods of preventing or treating a type 2 inflammatory disease in a subject in need thereof comprising administering an anti-alarmin binding molecule to the subject. In some embodiments, the type 2 inflammatory disease comprises asthma, viral exacerbations of allergic asthma, chronic rhinosinusitis with nasal polyps, allergic bronchopulmonary aspergillosis, atopic dermatitis, eosinophilic esophagitis, eosinophilic gastritis, eosinophilic gastroenteritis, eosinophilic colitis, allergic conjunctivitis, eosinophilia and food allergies. In some embodiments, the type 2 inflammatory disease is a viral-induced type 2 inflammatory disease selected from a group consisting of asthma, chronic obstructive pulmonary disease (COPD), eosinophilic esophagitis (EoE), chronic rhinosinusitis with nasal polyps (CRSwNP), and viral encephalitis, acute respiratory syndrome (SARS), Middle East respiratory syndrome (MERS), and COVID-19. In some embodiments, the type 2 inflammatory disease is a type 2 inflammatory disease associated with allergic exacerbations selected from a group consisting of asthma, allergic rhinitis, and atopic dermatitis. In some embodiments, the type 2 inflammatory disease is a type 2 inflammatory disease associated with environmental exacerbations selected from a group consisting of asthma, allergic rhinitis, and atopic dermatitis. In some embodiments, the type 2 inflammatory disease is a type 2 inflammatory disease associated with drug-induced exacerbations, wherein the drug is selected from non-steroidal anti-inflammatory drugs (NSAIDs), beta-blockers, ACE inhibitors, aspirin, and checkpoint inhibitors. In some embodiments, the treatment of a type 2 inflammatory disease results in a reduction in type 2 cytokine expression (e.g. IL 13, IL4 and IL5), and/or a reduction in eotaxin and eosinophils.

According to the present disclosure, the anti-alarmin binding molecule is administered as a single dose or as multiple doses. In some embodiments, the anti-alarmin binding molecule is selected from a group consisting of an anti-IL-25 antibody, an anti-IL-33 antibody, or an anti-TSLP antibody. In some embodiments, the anti-IL-33 antibody is selected from tozorakimab and itepekimab. In some embodiments, the anti-TSLP antibody is Tezepelumab.

According to the present disclosure, the anti-IL-25 antibody comprises a heavy chain variable domain comprising a HCDR1 of SEQ ID NO: 1, a HCDR2 of SEQ ID NO: 2, and a HCDR3 of SEQ ID NO: 3. In some embodiments, the anti-IL25 antibody comprises a heavy chain variable domain comprising the sequence of SEQ ID NO: 4. In some embodiments, the anti-IL-25 antibody comprises a heavy chain variable domain comprising a HCDR1 of SEQ ID NO: 9, a HCDR2 of SEQ ID NO: 10, and a HCDR3 of SEQ ID NO: 11. In some embodiments, the anti-IL25 antibody comprises a heavy chain variable domain comprising the sequence of SEQ ID NO: 12. In some embodiments, the anti-IL25 binding molecule comprises a light chain variable domain comprising a LCDR1 of SEQ ID NO: 5, a LCDR2 of SEQ ID NO: 6, and a LCDR3 of SEQ ID NO: 7. In some embodiments, the anti-IL25 binding molecule comprises a light chain variable domain comprising the sequence of SEQ ID NO: 8 or SEQ ID NO: 13. In some embodiments, the anti-IL-25 antibody comprises a heavy chain variable domain comprising a HCDR1 of SEQ ID NO: 1, a HCDR2 of SEQ ID NO: 2, and a HCDR3 of SEQ ID NO: 3, and a light chain variable domain comprising a LCDR1 of SEQ ID NO: 5, a LCDR2 of SEQ ID NO: 6, and a LCDR3 of SEQ ID NO: 7. In some embodiments, the anti-IL-25 antibody comprises a heavy chain variable domain comprising a HCDR1 of SEQ ID NO: 9, a HCDR2 of SEQ ID NO: 10, and a HCDR3 of SEQ ID NO: 11, and a light chain variable domain comprising a LCDR1 of SEQ ID NO: 5, a LCDR2 of SEQ ID NO: 6, and a LCDR3 of SEQ ID NO: 7. In some embodiments, the anti-IL-25 antibody comprises a heavy chain variable domain comprising the sequence of SEQ ID NO: 4 and a light chain variable domain comprising the sequence of SEQ ID NO: 8. In some embodiments, the anti-IL-25 antibody comprises a heavy chain variable domain comprising the sequence of SEQ ID NO: 12 and a light chain variable domain comprising the sequence of SEQ ID NO: 13.

According to the present disclosure, the anti-IL-25 antibody administered intraperitoneally, subcutaneously, or intravenously. In some embodiments, the therapeutically effective dose of the anti-IL-25 antibody according to the present disclosure is about 5 mg/kg, about 10 mg/kg, about 20 mg/kg, about 30 mg/kg, about 40 mg/kg, or about 50 mg/kg. In some embodiments, the therapeutic effective dose is administered about every week, about every two weeks, about every three weeks, or about every 4 weeks.

Also disclosed herein are methods of treating, inhibiting, reducing, decreasing, and/or ameliorating an inflammatory disease or condition or inflammation associated with a disease or condition (such as, for example, checkpoint inhibitor pneumonitis, fibrosis, a type 2 inflammatory disease, a rhinoviral infection, coronavirus infection, airway inflammation, rheumatoid arthritis, asthma, osteoarthritis, bone erosion, intraperitoneal abscesses and adhesions, inflammatory bowel disorder, allograft rejection, psoriasis, angiogenesis, atherosclerosis, cystic fibrosis and/or multiple sclerosis) comprising administering a therapeutic amount of any of the anti-alarmin binding molecules of any preceding aspect.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 illustrates the experiment protocol for evaluating the anti-fibrosis effects of anti-IL25 antibody.

FIG. 2 shows the body weights of mice of the naïve, isotype control (BLM+isotype control mAb), and LNR125 (BLM+LNR125) groups, respectively.

FIGS. 3A-3C show anti-fibrotic effects of LNR125 on BLM induced pulmonary fibrosis. FIG. 3A shows the lung index in mice of the naïve, isotype control (BLM+isotype control mAb), and LNR125 (BLM+LNR125) groups, respectively. On day 21, mice were sacrificed and the lungs weights were recorded in relation to the body weights of each mouse. FIG. 3B shows the measurements of microscopic lung collagen staining area. Two sets each per mouse of paraffin embedded slices of lung tissue were stained with Masson's Trichrome and the area of positive staining was analyzed with Imagel. FIG. 3C shows the fibrosis scores for mice of the naïve, isotype control (BLM+isotype control mAb), and LNR125 (BLM+LNR125) groups, respectively. Histopathological slides of lung tissue were scored for amount of fibrotic changes per sample. Lung fibrosis was assessed microscopically on a scale of 0-8 based on alveolar septae thickening, loss of normal architecture, and collagen deposition.

FIGS. 4A-4D show the profile of cells in bronchoalveolar lavage fluid (BALF). FIG. 4A shows the total cell count from BALF; FIG. 4B show the percentage of monocytes in relation to the total cell count in BALF: FIG. 4C shows the percentage of lymphocytes in relation to the total cell count in BALF; and FIG. 4D shows the percentage of neutrophils in relation to the total cell count in BALF from mice of the naïve, isotype control (BLM+isotype control mAb), and LNR125 (BLM+LNR125) groups, respectively.

FIGS. 5A-5F show histopathology of antibody treated pulmonary fibrosis. Sections of lung tissue were stained with either H&E (FIG. 5A-5C) or Masson's Trichrome (FIGS. 5D-5F). FIGS. 5A and 5D: Narive mice. FIGS. 5B and 5E: Isotype Control Mice. FIGS. 5C and 5F: LNR125 treated mice. The arrow indicates a foci of lymphocytes. Collagen deposition is indicated by staining in the Masson's Trichrome slides. All slides are shown at 40× magnification.

FIGS. 6A-6C shows the smoothness and thinness of the arteriole wall of the naïve (FIG. 6A) and LNR125 treated mice (FIG. 6B) versus the thicker arteriole wall of the isotype control group (FIG. 6).

FIGS. 7A-71 show immunohistochemistry of antibody treated pulmonary fibrosis. Lung samples were stained for the presence of IL-25 or its receptor, IL-17RB. The slides are shown at 40× magnification. FIGS. 7A-7C show IL-25 staining, FIGS. 7D-7F show IL-17 receptor B (IL-17RB) staining, and FIGS. 7G-71 show Masson's Trichrome staining of the samples from mice of the naïve, isotype control (BLM+isotype control mAb), and LNR125 (BLM+LNR 125) groups, respectively. The top arrow in FIG. 7B points to bronchiolar epithelial cells with intense IL-25 staining and the lower arrow denotes intercellular IL-25 staining adjacent to an area of collagen. The asterisks shows fibroblasts. The arrow in FIG. 7C shows a macrophagc with IL-25 staining.

FIG. 8 shows the representative IL-17RB staining of lung samples from non-diseases control and a patient with IPF.

FIGS. 9A-9D show the expression of IL-25 protein by alveolar epithelial cells (A549 cells) stressed with etoposide/H2O2.

FIGS. 10A and 10B show that blockade of IL25 by LNR 125 reduces cell proliferation of MRC-5 fibroblasts stimulated with conditioned medium (CM) from H202/etoposide stressed alveolar epithelial cells (A549 cells). The cell numbers of MRC-5 fibroblasts in the condition medium (CM) from H2O2/etoposide treated with anti-IL25 antibody or isotype control for 24 hours (FIG. 10A) and 48 hours (FIG. 10B), respectively. DMEM is media from non-stressed A549 cells.

FIGS. 11A and 11B show that blockade of IL25 by LNR 125 prevents CM-induced expression of pro-fibrotic mediator fibronectin by MRC-5 fibroblasts for 24 hours (FIG. 11A) and 48 hours (FIG. 11B), respectively.

FIGS. 12A-12D show that blockade of IL25 by LNR 125 reduces CM-induced collagen I (Col1A1) & III (Col3A 1) gene expression in MRC-5 fibroblasts for 24 hours (FIGS. 12A and 12C) and 48 hours (FIGS. 12B and 12D), respectively.

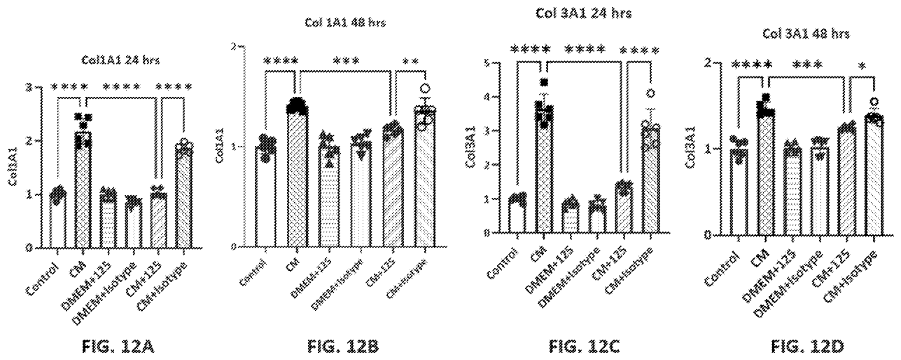

FIG. 13 illustrates the experimental protocol for evaluating the prevention of pneumonitis by anti-IL25 antibody LNR125 in a murine model of checkpoint inhibitor-induced adverse events.

FIG. 14 shows that mice administered the immune checkpoint inhibitors (ICIs) anti-PD-1 and anti-CTLA-4 have increased severity of leukocyte infiltration in the liver, lungs, heart, colon, and pancreas and that treatment with anti-IL25 antibody LNR125 reduces immune cell infiltration in these organs. LNR 125 HD and LNR 125 LD=20 and 10 mg/kg, respectively.

### DETAILED DESCRIPTIONS

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example. “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable. i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type 1 diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

As used herein the term “binding molecule” refers to any immunotoxin or immunoglobulin including monoclonal antibodies, polyclonal antibodies, chimeric antibodies, diabodies, nanobodies, humanized or human antibodies, as well as antibodies fragments and functional variants including antigen-binding and/or variable domain comprising fragment of an immunoglobulin that competes with the intact immunoglobulin for specific binding to the binding partner of the immunoglobulin.

The term “alarmin” refers to an array of structurally diverse multifunctional host proteins that are rapidly released during infection or tissue damage, and that have mobilizing and activating effects on receptor-expressing cells engaged in host defense and tissue repair.

As used herein the term “anti-alarmins” or “anti-alarmin binding molecules” refer to molecules that can inhibit or counteract the effects of alarmins. For example, the molecules may bind to alarmins and neutralize the activity of alarmins. Anti-alarmin antibodies refer to any antibodies against alarmins and includes neutralizing anti-alarmin antibodies.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. Also included within the meaning of “antibody or fragments thereof” are conjugates of antibody fragments and antigen binding proteins (single chain antibodies). As used herein, the term “antibody” or “antibodies” can also refer to a human antibody and/or a humanized antibody.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

“Pathogen” is an agent that causes infection or disease, especially a virus, bacterium, fungus, protozoa, or parasite.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Anti-Alarmin Binding Molecules

The role of type-2 immunity has been recognized by the development of biologics (monoclonal antibodies mAbs) that inhibit interleukin 4 (IL-4), IL-5, or IL-13 and reduce frequency of exacerbations up to 50%. More recently focus has shifted to airway epithelial cell-expressed alarmins such as TSLP and IL-33 that stimulate type 2 immune pathways. IL-25 is also expressed by epithelial cells and stimulates type-2 inflammation. IL-25 expression is higher at baseline and during RV infection in individuals with asthma. IL-25 signals through an IL-17RA/IL-17RB heterodimer receptor on immune cells such as type-2 innate lymphoid cells (ILC2), T helper 2 (Th2) cells, eosinophils, basophils, mast cells as well as bronchial epithelial cells (BECs) which constitutively express IL-25 for immediate secretion upon exposure to proteases or pathogens.

In some embodiments, the present disclosure provides methods of preventing or treating pneumonitis (including checkpoint inhibitor induced pneumonitis), fibrosis, and type 2 inflammatory disorders comprising administration of an anti-alarmin binding molecule to a subject in need thereof. Alarmins are endogenous molecules that are released by cells in response to tissue damage, infection, or other forms of stress. They act as “danger signals” that alert the immune system to the presence of a potential threat and trigger an inflammatory response. Alarmins can be released from a variety of cell types, including damaged or dying cells, epithelial cells, endothelial cells, as well as immune cells such as macrophages, dendritic cells and neutrophils. They can be found in various bodily fluids, such as blood, cerebrospinal fluid, and synovial fluid.

There are many types of alarmins. including cytokines, Damage-Associated Molecular Patterns (DAMPs), and extracellular matrix molecules. Alarmin cytokines are small proteins that are released by cells and act as signaling molecules in the immune system. Examples include IL-1α, IL-1β, and IL-33, IL-25, and thymic stromal lymphopoietin (TSLP). DAMPs are molecules that are released from damaged cells and act as danger signals. Examples include HMGB1 and ATP. Extracellular matrix molecules are molecules that are released from the extracellular matrix of cells and can activate immune cells. Examples include hyaluronan and heparan sulfate.

Alarmins play an important role in the immune response by alerting the immune system to the presence of a potential threat and triggering an inflammatory response. However, excessive or prolonged activation of the immune system by alarmins can lead to tissue damage and chronic inflammation, which is associated with a range of diseases such as autoimmune disorders, cancer, and neurodegenerative diseases. Therefore, alarmins are potential therapeutic targets for various inflammatory and autoimmune diseases. Such therapeutics include anti-IL25 binding molecules, anti-IL-33 binding molecules, and anti-TSLP binding molecules.

In some embodiments, the present disclosure provides a method of treating fibrosis in a subject in need thereof comprising administering an anti-alarmin binding molecule to the subject. In some embodiments, the present disclosure provides methods of treating pneumonitis in a subject in need thereof comprising administering an anti-alarmin binding molecule to the subject. In some embodiments, the present disclosure provides a method of treating checkpoint inhibitor induced pneumonitis in a subject in need thereof comprising administering an anti-alarmin binding molecule to the subject. In some embodiments, the anti-alarmin binding molecule is an anti-IL25 binding molecule, an anti-IL-33 binding molecule, or an anti-TSLP binding molecule.

### Anti-IL-33 Binding Molecules

In some embodiments, the present disclosure provides methods of preventing or treating pneumonitis (including checkpoint inhibitor induced pneumonitis), fibrosis, and type 2 inflammatory disorders comprising administration of an anti-IL-33 binding molecule to a subject in need thereof. In some embodiments, the anti-IL-33 binding molecule is an anti-IL-33 antibody.

Interleukin 33 (IL-33) is a multifunctional cytokine, a new member of the IL-1 family. Encoded by the IL-33 gene, it is constitutively expressed in structural cells such as smooth muscle cells, epithelial cells, and endothelial cells. In macrophages and dendritic cells, IL-33 can be induced by inflammatory factors. IL-33 is a ligand for ST2, a toll-like/interleukin-1 receptor super-family member that associates with an accessory protein, IL-1 RAcP (Kakkar and Lee, Nature Reviews—Drug Discovery 7(10):827-840 (2008), Schmitz et al., Immunity 23:479490 (2005): Liew et al., Nature Reviews—Immunology 10:103-110 (2010); US 2010/0260770; US 2009/0041718). Upon activation of ST2/IL-1RAcP by IL-33, a signaling cascade is triggered through downstream molecules such as MyD88 (mycloid differentiation factor 88) and TRAF6 (TNF receptor associated factor 6), leading to activation of NFcB (nuclear factor-KB), among others. IL-33 signaling has been implicated as a factor in a variety of diseases and disorders. (Liew et al., Nature Reviews—Immunology 10:103-110 (2010))

In some embodiments, the anti-IL-33 antibody is selected from tozorakimab (AstraZeneca) and itepekimab (Regeneron). In some embodiments, the present disclosure provides an antibody that targets IL-33 receptor, ST2 In some embodiments, the anti-ST2 antibody is selected from astegolimab (Roche). Details of additional exemplary IL-33 antibodies are disclosed in US20210363236, WO2021204707, and WO2022063281, each of which are incorporated by reference for any purpose.

### Anti-TSLP Binding Molecules

In some embodiments, the present disclosure provides methods of preventing or treating pneumonitis (including checkpoint inhibitor induced pneumonitis), fibrosis, and type 2 inflammatory disorders comprising administration of an anti-TSLP binding molecule to a subject in need thereof. In some embodiments, the anti-TSLP binding molecule is an anti-TSLP antibody.

Full length TSLP is a short-chain four α-helical bundle cytokine that induces Signal Transducer and Activator of Transcription (STAT5) phosphorylation via the functional TSLP receptor (TSLPR), a heterodimeric receptor complex consisting of the IL-7Rα and the unique TSLPR chain (CRFL2) (Park et al, JEM 192(5):659-682 (2002)). In addition a short isoform of TSLP (sfTSLP) expressed from an alternative transcription start site appears to be expressed in human cells, but does not appear to activate STAT5 and may serve a different function to full length TSLP (Bjerkan et al., Mucosal immunology 8(1) 49-56 (2015)). TSLP is most highly produced by epithelial and stromal cells lining the barrier surfaces of the skin, gut, and lungs but is also produced by other cell types implicated in allergic disease (e.g., dendritic cells, mast cells, smooth muscle cells). Production is induced upon exposure to a number of factors including protease allergens (Kouzaki et al, J Immunol. 183(2):1427-34 (2009)), viruses, bacteria, inflammatory mediators, cigarette smoke and environmental particulates (Bleck et al, J Clin Immunol 28(2):147-156 (2008)). TSLP acts on a broad range of cell types (e.g. dendritic cells, CD4+ T cells, eosinophils, basophils, mast cells and Type 2 innate lymphoid cells (ILC2) (Mjosberg et al, Immunity 37(4):649-59 (2012)) to drive inflammation, and in particular, Type 2 inflammation (characterised by the production of the cytokines IL-5, IL-13 and IL-4. Type 2 inflammation is a feature of asthma and other allergic diseases such as atopic dermatitis and Netherton Syndrome. TSLP has been found to induce fibroblast accumulation and collagen deposition in animals demonstrating an additional role in promoting fibrotic disorders.

In some embodiments, the anti-TSLP antibody is Tezepelumab (AstraZeneca/Amgen). Details of additional exemplary anti-TSLP antibodies are described in US20160264658, WO2022226339, US20220363781, and US20210121406, each of which are incorporated by reference for any purpose.

### Anti-IL-25 Antibodies

In some embodiments, the present disclosure provides methods of preventing or treating pneumonitis (including checkpoint inhibitor induced pneumonitis), fibrosis, and type 2 inflammatory disorders comprising administration of an anti-IL-25 binding molecule to a subject in need thereof. In some embodiments, the anti-IL-25 binding molecule is an anti-IL-25 antibody.

IL-25 is a cytokine that is structurally related to interleukin-17 (IL-17) and is sometimes referred to as IL-17E. It is a secreted, homodimeric glycoprotein that interacts with and signals through the heterodimeric IL-17RB/IL-17RA receptor (Iwakura, et.al., Immunity, 34:149 (2010)). IL-25 is produced by Th2 cells, epithelial cells, endothelial cells, alveolar macrophages, mast cells, eosinophils and basophils (Rouvier, E. et.al., J. Immunol. 150:5445-5456 (1993); Pan, G. et.al., J. Immunol. 167:6559-6567 (2001); Kim, M. et.al., Blood, 100:2330-2340 (2002)). Signaling through IL-25 is associated with eosinophil recruitment, initiation of Th2 and Th9 responses and suppression of Th1 and Th17 cell responses. IL-25 induces the production of other cytokines, including IL-4, IL-5 and IL-13, in multiple tissues (Fort, M et.al., Immunity 15:985-995 (2001)). IL-25 has been implicated in chronic inflammation associated with the gastrointestinal tract and the IL-25 gene has been identified in a chromosomal region associated with autoimmune diseases of the gut, such as inflammatory bowel disease (IBD) (Buning, C. et.al., Eur. J. Immunogener. Oct; 30(5): 329-333 (2003)). IL-25 has also been shown to be upregulated in samples from patients with asthma (Sherkat, R. et.al., Asia Pac. Allergy Oct; 4(4):212-221 (2014)). Accordingly, blockade of IL-25 signaling may be useful for the treatment of various disorders associated with IL-25 activity or expression.

In one aspect, disclosed herein are isolated anti-IL25 binding molecules comprising a heavy chain variable domain comprising a HCDR1. HCDR2, and HCDR2 as set forth in SEQ ID NO: 1, SEQ ID NO: 2, and SEQ ID NO: 3, respectively (such as, for example, an anti-IL-25 binding molecule comprising a heavy chain variable domain as set forth in SEQ ID NO: 4). In some aspects, the heavy chain variable domain may comprise a substitution at residue 29 in CDR1, residue 64 in CDR2. and/or residue 105 in CDR3. For example, the substitution can comprise an asparagine to serine substitution (N29S) as set forth in SEQ ID NO: 9, an alanine to serine substitution (A64S) as set forth in SEQ ID NO: 10, and/or Phenylalanine to Histidine substitution (F105H) as set forth in SEQ ID NO: 11. To get rid of hydrophobicity which is causing dimerization during elution at pH 2.5, a library of amino acid-changing mutations was introduced at F105 to determine whether potency could be retained in the absence of dimerization and under potentially less harsh (pH3.5) elution conditions. Of the amino acids tested at that position, only one change, F105H, allowed for the production of 99% monomer and retention of activity. The introduced histidine likely disrupts the hydrophobic interactions that may have been occurring between the heavy chain CDR3s of two different antibody molecules. In one embodiment, an anti-IL-25 binding molecule comprises a heavy chain variable domain as set forth in SEQ ID NO: 12. The substitution A64S was introduced when the heavy chain was humanized onto the IGHV2 human germline. Also disclosed herein are isolated anti-IL25 binding molecules, further comprising a light chain variable domain comprising a CDR1, CDR2, and CDR2 as set forth in SEQ ID NO: 5, SEQ ID NO: 6, and SEQ ID NO: 7, respectively (such as, for example, an anti-IL-25 binding molecule comprising a light chain variable domain as set forth in SEQ ID NO: 8 or SEQ ID NO: 13). In some instances, the light chain variable domain can comprise a substitution at residue 105 from a leucine to a valine (L105V) as set forth in SEQ ID NO: 13. Table 1 provides the sequences of exemplary anti-IL25 antibodies.

TABLE 1
Sequences of exemplary anti-IL25 antibodies

| | | SEQ ID |
|---|---|---|
| Domain or Region | Sequence | NO |
| LNR125 HCDR1 | NTSGMGVG | 1 |
| LNR125 HCDR2 | HIWWDDVKRYNPALKS | 2 |
| LNR125 HCDR3 | TLPHFFDY | 3 |
| LNR125 VH | QVTLKVSGPGILQPSQTLSLTCSFSGFSLNTSGMGV | 4 |
| | GWIRQPSGKGLEWLAHIWWDDVKRYNPALKSRLT | |
| | ISKDTSGSQVFLKIASVDTADTATYYCARTLPHFFD | |
| | YWGQGTTLTVSS | |
| LNR125 LCDR1 | RASSSVSYMY | 5 |
| LNR125 LCDR2 | RTSNLAS | 6 |
| LNR125 LCDR3 | KQYHSYPPTWT | 7 |
| LNR125 VL | DIQMTQSPAIMSASPGEKVTISCRASSSVSYMYWYQ | 8 |
| | QKSGSSPKPWIYRTSNLASGVPARFSGSGSGTSYSL | |
| | TISSMEAEDAATYYCKQYHSYPPTWTFGGGTKLEI | |
| | KR | |
| LNR125.38 | STSGMGVG | 9 |
| HCDR1 | | |
| LNR125.38 | HIWWDDVKRYNPSLKS | 10 |
| HCDR2 | | |
| LNR125.38 | TLPHFHDY | 11 |
| HCDR3 | | |
| LNR125.38 | QVTLKESGPTLVKPTQTLTLTCTFSGFSLSTSGMGV | 12 |
| VH | GWIRQPPGKALEWLAHIWWDDVKRYNPSLKSRLTI | |
| | TKDTSGSQVVLTMTNMDPVDTATYYCARTLPHFH | |
| | DYWGQGTTLTVSS | |
| LNR125.38 | RASSSVSYMY | 5 |
| LCDR1 | | |
| LNR125.38 | RTSNLAS | 6 |
| LCDR2 | | |
| LNR125.38 | KQYHSYPPTWT | 7 |
| LCDR3 | | |
| LNR125.38 | DIQMTQSPSTLSASVGDRVTITCRASSSYSYMYWY | 13 |
| VL | QQKPGKAPKPLIYRTSNLASGVPSRFSGSGSGTEYT | |
| | LTISSLQPDDFATYYCKQYHSYPPTWTFGGGTKVEI | |
| | KR | |

In the variable domains, CDR1, CDR2 and CDR3 (from left to right) sequences are underlined.

In some embodiments, the anti-IL-25 antibody LNR125 comprises a heavy chain variable domain comprising a HCDR1 of SEQ ID NO: 1, a HCDR2 of SEQ ID NO: 2, and a HCDR3 of SEQ ID NO: 3, and a light chain variable domain comprising a LCDR1 of SEQ ID NO: 5, a LCDR2 of SEQ ID NO: 6, and a LCDR3 of SEQ ID NO: 7. In some embodiments, the humanized anti-IL-25 antibody LNR 125. 38 comprises a heavy chain variable domain comprising a HCDR1 of SEQ ID NO: 9, a HCDR2 of SEQ ID NO: 10, and a HCDR3 of SEQ ID NO: 11, and a light chain variable domain comprising a LCDR 1 of SEQ ID NO: 5, a LCDR2 of SEQ ID NO: 6, and a LCDR3 of SEQ ID NO: 7. In some embodiments, the anti-IL-25 antibody LNR125 comprises a heavy chain variable domain comprising the sequence of SEQ ID NO: 4 and a light chain variable domain comprising the sequence of SEQ ID NO: 8. In some embodiments, the humanized anti-IL-25 antibody LNR125.38 comprises a heavy chain variable domain comprising the sequence of SEQ ID NO: 12 and a light chain variable domain comprising the sequence of SEQ ID NO: 13. Humanized anti-IL-25 antibodies as disclosed herein exhibit reduced immunogenicity in human being. Table 2 provides the potency and binding affinity of anti-IL25 antibodies LNR125 and LNR125.38. Human IL-25 for potency and affinity measurement is made from human (HEK) sources; mouse IL-25 for potency and affinity measurement is made from mouse (NSO) source.

TABLE 2
Potency and binding affinity of exemplary anti-IL25 antibodies

| | Potency | | | | | | |
|---|---|---|---|---|---|---|---|
| | IC50 in HT29 cell assay | | | | | | |
| | (mg/ml) | | Dissociation Constants (KD) (pM) | | | Protein | |
| Anti-IL-25 | Human | Mouse | Human | Mouse | Cyno Monkey | Yield | |
| mAbs | (HEK) | (NSO) | (HEK) | (NSO) | (HEK) | (CHO) | |
| LNR125 | 0.12 | 0.13 | 128 | 168 | 170 | 10 | mg/L |
| LNR125.38 | 0.10 | 0.78 | 51 | 96 | 235 | 96 | g/L |

Additional anti-IL-25 antibodies are disclosed in U.S. Pat. Nos. 8,785,605, 8,658,169, 8,206,717, and 9,840,557; and PCT publications WO2011123507, WO2010/038155 and WO2008/129263, each of which are incorporated by reference for any purpose.

### Methods of Use

In some embodiments, the present disclosure provides a method of preventing or treating fibrosis in a subject in need thereof comprising administering an anti-alarmin binding molecule to the subject. In some embodiments, the present disclosure provides methods of preventing or treating pneumonitis in a subject in need thereof comprising administering an anti-alarmin binding molecule to the subject. In some embodiments, the present disclosure provides a method of preventing or treating checkpoint inhibitor induced pneumonitis in a subject in need thereof comprising administering an anti-alarmin binding molecule to the subject. In some embodiments, the present disclosure provides a method of preventing or treating a type 2 inflammatory disease in a subject in need thereof comprising administering an anti-alarmin binding molecule to the subject. In some embodiments, the anti-alarmin binding molecule is an anti-IL25 binding molecule, an anti-IL-33 binding molecule, or an anti-TSLP binding molecule.

### Pneumonitis

Pneumonitis is an inflammatory condition that affects the lung tissue. Pneumonitis can be caused by a variety of factors, including exposure to interstitial lung disease, viral infection, autoimmune disease, allergy, inhalation of occupational debris, dusts, fibers, fumes or vapors, inhalation of chemicals or molds, sepsis, adverse reaction to medications, aspirin overdose, hypersensitivity to environmental antigens, overexposure to chlorine, exposure to herbicides, fluorocarbons, radiation, chemotherapy, and/or treatment with one or more immune checkpoint inhibitors. Patients with chronic obstructive pulmonary disease (COPD), ulcerative colitis (UC), lung cancer, gastrointestinal (GI) cancer, pulmonary fibrosis, atopic dermatitis, asthma, or eosinophilic esophagitis (EOE) may have high risk of developing pneumonitis.

Pneumonitis is typically associated with an inflammatory response in the lungs, which can lead to tissue damage and impaired lung function. Alarmins play a key role in initiating and amplifying the inflammatory response in the lungs, thus targeting these molecules with neutralizing antibodies against alarmins may help to reduce inflammation and prevent further damage.

In some embodiments, the present disclosure provides methods of preventing or treating pneumonitis in a subject in need thereof comprising administering an anti-alarmin binding molecule to the subject. In some embodiments, the anti-alarmin binding molecule is selected from an anti-IL25 antibody, an anti-IL-33 antibody, or an anti-TSLP antibody described herein.

In some embodiments, the anti-alarmin binding molecules are administered to the subject prior to the onset of pneumonitis symptoms in order to prevent one or more symptoms of pneumonitis. In some embodiments, the anti-alarmin binding molecules are administered to the subject after the onset of pneumonitis symptoms in order to treat one or more symptoms of pneumonitis. In some embodiments, the anti-alarmin binding molecules are administered to the subject 1, 2, 3, 4, 5, 6, 7, 8, 9, 10 or more times. In some embodiments, the anti-alarmin binding molecules are administered to the subject once a week for 1, 2, 3, 4, 5, or more weeks after the onset of symptoms.

### Checkpoint Inhibitor Induced Pneumonitis

Checkpoint inhibitor pneumonitis (CIP) is an inflammatory lung condition that can occur as a side effect of cancer immunotherapy with checkpoint inhibitors. Checkpoint inhibitors work by targeting proteins on immune cells that normally act as “checkpoints” to prevent the immune system from attacking healthy cells. While checkpoint inhibitors can be effective in treating cancer, they can also cause an overactive immune response that can lead to inflammatory side effects such as pneumonitis. Without being bound by a theory, it is thought that checkpoint inhibitors may trigger an immune response that is too strong or too widespread, leading to inflammation and tissue damage in the lungs. It is believed that checkpoint inhibitors can affect various immune cells and signaling pathways, including T cells, B cells, cytokines, and chemokines.

Non-limiting examples of checkpoint inhibitors may include anti-programmed death receptor-1 (PD1) molecules (e.g. anti-PD1 antibody or fusion proteins that target PD1), anti-Programmed Death Ligand 1 (PD-L1) molecules (e.g. anti-anti-PD-L1 antibody or fusion proteins that target PD-L1 programmed death receptor-1), anti-cytotoxic T-lymphocyte associated protein 4 (CTLA4) molecules (e.g. anti-CTLA4 antibody or fusion proteins that target CTLA4), anti-Lymphocyte-activation gene 3 (LAG3) molecules (e.g. anti-LAG3 antibodies or fusion proteins that target LAG3), anti-T-cell immunoreceptor with Ig and ITIM domains (TIGIT) molecules (e.g. anti-TIGIT antibodies or fusion proteins that target TIGIT), anti-T-cell immunoglobulin and mucin domain-containing protein 3 (TIM-3) molecules (e.g. anti-TIM-3 antibodies or fusion proteins that target TIM-3), anti-V-domain Ig suppressor of T cell activation (VISTA) molecules (e.g. anti-VISTA antibodies or fusion proteins that target VISTA), anti-B and T lymphocyte attenuator (BTLA) molecules (e.g. anti-BTLA antibodies or fusion proteins that target BTLA), anti-Sialic acid-binding Ig-like lectin 15 (Siglec-15) molecules (e.g. anti-Siglec-15 antibodies or fusion proteins that target Siglec-15), and anti-CD96 molecules (e.g. anti-CD96 antibodies or fusion proteins that target CD96). In some embodiments, the anti-PD1 antibody is selected from pembrolizumab, nivolumab, cemiplimab, dostarlimab, spartalizumab. camrelizumab, sintilimab, tislelizumab, toripalimab, and retifanlimab. In some embodiments, the anti-CTLA4 antibody is selected from ipilimumab, tremelimumab, BMS-986249, quavonlimab, and AGEN1884. In some embodiments, the anti-PD-L1 antibody is selected from atezolizumab, avelumab. and durvalumab.

In some embodiments, the checkpoint inhibitor may be delivered by T cells such as CAR-T cells, allogeneic T cells to enhance therapeutic effectiveness. In some embodiments, the checkpoint inhibitor is delivered by CAR-T or allogeneic T cells that express a checkpoint inhibitor polypeptide. CAR-T therapy involves genetically modifying a patient's T cells to recognize and attack cancer cells, and this approach has shown promise in treating certain types of cancer. By combining CAR-T therapy with checkpoint inhibitors, it may be possible to further enhance the immune response and improve outcomes for patients. Similarly, allogeneic T cell therapy, which involves using T cells from a healthy donor to target cancer cells, may also be used in combination with checkpoint inhibitors to improve outcomes for patients.

In some embodiments, the checkpoint inhibitor is delivered by viral vectors (i.e. checkpoint inhibitor gene therapy). One approach to checkpoint inhibitor gene therapy involves genetically modifying immune cells, such as T cells or natural killer cells, to express checkpoint inhibitors on their surface. By doing so, these cells can more effectively recognize and attack cancer cells, and can help to overcome the immunosuppressive effects of the tumor microenvironment. Another approach to checkpoint inhibitor gene therapy involves delivering genes encoding checkpoint inhibitors directly to tumor cells, either through viral vectors or other delivery systems. This approach is designed to “reprogram” the tumor cells to express checkpoint inhibitors, which can help to stimulate an immune response against the tumor. For example, a AAV-anti-PD1 vector may be delivered to produce monoclonal antibodies in tumor cells

In some embodiments, the checkpoint inhibitor antibody may be conjugated to a therapeutic moiety such as, for example, a cytotoxin, a therapeutic agent (e.g., a chemotherapy drug or an immunosuppressant), a radioisotope, an ultrasound sensitizer, or an exosome secretion inhibitor. When conjugated to cytotoxins, these antibody conjugates are referred to as “immunotoxins”. A cytotoxin or cytotoxic agent includes any agent that is detrimental to (e.g., kills) cells. Non-limiting examples include taxol, cytochalasin B, gramicidin D. ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. A therapeutic agent include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). A checkpoint inhibitor antibody of the present disclosure can be conjugated to a radioisotope, e.g., radioactive iodine, to generate cytotoxic radiopharmaceuticals for treating a related disorder, such as a cancer. In some embodiments, a checkpoint inhibitor antibody of the present disclosure can be conjugated to an ultrasound sensitizer selected from the group consisting of porphyrins, porphyrin isomers and expanded porphyrins to specifically responds to ultrasonic waves for diagnosing and treating cancer. Details of immune checkpoint inhibitor conjugated with an ultrasonic sensitizer are described in WO2022124664, the contents of which are incorporated by reference for any purpose. In some embodiments, a checkpoint inhibitor antibody of the present disclosure can be conjugated to an exosome secretion inhibitor selected from the group consisting of Manumycin A, GW4869, cannabidiol and endothelin receptor antagonists. Details of immune checkpoint inhibitor conjugated with an exosome secretion inhibitor are described in WO2021153981. the contents of which are incorporated by reference for any purpose.

In some embodiments, the present disclosure provides methods of preventing or treating checkpoint inhibitor induced pneumonitis in a subject in need thereof comprising administering an anti-alarmin binding molecule to the subject. In some embodiments, the anti-alarmin binding molecule is selected from an anti-IL25 antibody, an anti-IL-33 antibody, or an anti-TSLP antibody described herein.

In some embodiments, the anti-alarmin binding molecule is administered to the subject prior to the administration of one or more checkpoint inhibitors in order to prevent checkpoint inhibitor induced pneumonitis. In some embodiments, the anti-alarmin binding molecule is administered to the subject at least 1, 2, 3, 4, 5, 6, 7, 8, 9, 10 or more days prior to the administration of one or more checkpoint inhibitors.

In some embodiments, the anti-alarmin binding molecule is administered to the subject concomitantly with the administration of one or more checkpoint inhibitors in order to prevent checkpoint inhibitor induced pneumonitis. In some embodiments, the anti-alarmin binding molecule and the one or more checkpoint inhibitors are formulated in the same composition. In some embodiments, anti-alarmin binding molecule and the one or more checkpoint inhibitors are formulated in separate compositions and administered sequentially in the same treatment.

In some embodiments, the anti-alarmin binding molecule is administered to the subject after the administration of one or more checkpoint inhibitors, but before the onset of pneumonitis symptoms, in order to prevent checkpoint inhibitor induced pneumonitis. In some embodiments, the anti-alarmin binding molecule is administered within 24 hours after the administration of the one or more checkpoint inhibitors and before the onset of symptoms. In some embodiments, the anti-alarmin binding molecule is administered 24 hours or more after the administration of the one or more checkpoint inhibitors and before the onset of symptoms. In some embodiments, the anti-alarmin binding molecule is administered 1, 2, 3, 4, 5, 6, 7, 8, 9, 10, 11, 12, 13, or 14 days after administration of the one or more checkpoint inhibitors and before the onset of symptoms. In some embodiments, the anti-alarmin binding molecule is administered 1, 2, 3, 4, 5, 6, 7, 8, 9, 10, 11, 12, 13, or 14 weeks after administration of the one or more checkpoint inhibitors and before the onset of symptoms.

In some embodiments, the anti-alarmin binding molecule is administered to the subject after the administration of one or more checkpoint inhibitors and after the onset of pneumonitis symptoms in order to treat checkpoint inhibitor induced pneumonitis. In some embodiments, the anti-alarmin binding molecule is administered within 24 hours after the onset of symptoms. In some embodiments, the anti-alarmin binding molecule is administered 24 hours or more after the onset of symptoms. In some embodiments, the anti-alarmin binding molecule is administered 1, 2, 3, 4, 5, 6, 7, 8, 9, 10, 11, 12, 13, or 14 days after the onset of symptoms. In some embodiments, the anti-alarmin binding molecule is administered 1, 2, 3, 4, 5, 6, 7, 8, 9, 10, 11, 12, 13, or 14 weeks after the onset of symptoms.

### Fibrosis

Fibrosis is a medical condition in which there is an excessive build-up of scar tissue in an organ or tissue, leading to a loss of function. The fibrosis process occurs when there is prolonged inflammation or injury to the affected tissue, leading to the formation of excess collagen and other extracellular matrix components. This scar tissue can accumulate over time and can cause the tissue to become stiff and less functional.

There are several types of fibrosis, which can affect different organs and tissues in the body. Some of the most common types of fibrosis include pulmonary fibrosis, liver fibrosis, cardiac fibrosis, renal fibrosis, skin fibrosis, gastrointestinal fibrosis, colon fibrosis, and pancreatic fibrosis. Pulmonary fibrosis affects the lungs and can lead to shortness of breath, cough, and difficulty breathing. Causes of pulmonary fibrosis include exposure to environmental toxins, autoimmune disorders, and infections. Liver fibrosis affects the liver and can lead to cirrhosis, liver failure, and other complications. Causes of liver fibrosis include alcohol abuse, viral hepatitis, and metabolic disorders. Cardiac fibrosis affects the heart and can lead to heart failure and other cardiovascular complications. Causes of cardiac fibrosis include hypertension, aging, and inflammation. Renal fibrosis affects the kidneys and can lead to kidney failure and other complications. Causes of renal fibrosis include diabetes, hypertension, and glomerulonephritis. Skin fibrosis affects the skin and can lead to disfigurement and impaired function. Causes of skin fibrosis include scleroderma and other autoimmune disorders. Gastrointestinal fibrosis affects the digestive system and can lead to obstruction and other complications. Causes of gastrointestinal fibrosis include Crohn's disease and other inflammatory bowel diseases. Colon fibrosis is a condition in which the normal tissue of the colon is replaced by fibrotic scar tissue. This can occur as a result of chronic inflammation in the colon, such as in inflammatory bowel disease (IBD), or due to other causes. Pancreatic fibrosis is a condition in which the normal pancreatic tissue is replaced by fibrotic scar tissue. This can occur as a result of chronic inflammation in the pancreas, which can be caused by conditions such as chronic pancreatitis or pancreatic cancer.

Some common causes of fibrosis include chronic diseases, infections, toxins, and physical trauma. Fibrosis can lead to serious health problems, depending on the organ or tissue affected. In the lungs, for example, fibrosis can cause shortness of breath, coughing, and decreased lung function, and can lead to conditions such as idiopathic pulmonary fibrosis (IPF) or interstitial lung disease (ILD). In the liver, fibrosis can lead to cirrhosis and liver failure.

Fibrosis formation is the result of the interactions between many cell types and various fibrogenic growth factors/cytokines in a pro-fibrotic environment. The mediators of fibrosis common in different tissues include pro-fibrotic cells and pro-fibrotic molecules (growth factors/cytokines), as well as other influences such as from the extracellular matrix (ECM), mechanical tension, or oxidative stress.

Non-limiting examples of molecular pro-fibrotic mediators that can contribute to this process, including transforming growth factor beta (TGF-β), connective tissue growth factor (CTGF), platelet-derived growth factor (PDGF), fibroblast growth factor (FGF), endothelin-1 (ET-1), Th2-type cytokines (IL-4, IL-5, IL-13, IL-21), chemokines (MCP-1, MIP-1β), angiogenic factors (VEGF), TNF-α, peroxisome proliferator-activated receptors (PPARs), acute phase proteins (SAP), caspases, components of the renin-angiotensin-aldosterone system (Angiotensin II/ANG II), and endothelin (ET).

The microenvironment of injured or diseased tissues can also be critical to inducing fibrosis. Excluding molecular pro-fibrotic factors, the environmental factors involved with the up-regulation of fibrosis may include: vascular damage, inflammation, oxidative stress, ECM, and mechanical tension. For tissue engineering materials, certain characteristics of exogenous biomaterials are often related with fibrosis formation.

In some embodiments, the present disclosure provides methods of preventing or treating fibrosis in a subject in need thereof comprising administering an anti-alarmin binding molecule to the subject. In some embodiments, the fibrosis is idiopathic pulmonary fibrosis or bleomycin-induced fibrosis. In some embodiments, the fibrosis is the result of interstitial lung disease, viral infection, autoimmune disease, allergy, inhalation of occupational debris, dusts, fibers, fumes or vapors, inhalation of chemicals or molds, sepsis, adverse reaction to medications, aspirin overdose, hypersensitivity to environmental antigens, overexposure to chlorine, exposure to herbicides, fluorocarbons, radiation, chemotherapy, immune dysregulation, and/or treatment with one or more immune checkpoint inhibitors. Immune dysregulation refers to an abnormal or dysfunctional immune response, which can result in a range of conditions and diseases. In some cases, immune dysregulation can lead to fibrosis, a process in which normal tissue is replaced by fibrotic scar tissue. There are many different ways in which immune dysregulation can lead to fibrosis. For example, chronic inflammation can lead to the recruitment of immune cells and the production of cytokines and growth factors that stimulate fibroblast activity and collagen deposition, resulting in fibrosis. Similarly, persistent activation of the immune system can lead to the overproduction of extracellular matrix proteins, leading to fibrosis. Non-limiting examples of conditions and diseases that are associated with immune dysregulation and fibrosis include rheumatoid arthritis, systemic sclerosis, idiopathic pulmonary fibrosis, and inflammatory bowel disease.

In some embodiments, the present disclosure provides methods of preventing or treating fibrosis in a subject in need thereof comprising administering an anti-alarmin binding molecule to the subject. In some embodiments, the anti-alarmin binding molecule is selected from an anti-IL25 antibody, an anti-IL-33 antibody, or an anti-TSLP antibody described herein.

In some embodiments, the anti-alarmin binding molecules are administered to the subject prior to the onset of fibrosis symptoms in order to prevent one or more symptoms of fibrosis. In some embodiments, the anti-alarmin binding molecules are administered to the subject after the onset of fibrosis symptoms in order to treat one or more symptoms of fibrosis. In some embodiments, the anti-alarmin binding molecules are administered to the subject 1, 2, 3, 4, 5, 6, 7, 8, 9, 10 or more times. In some embodiments, the anti-alarmin binding molecules are administered to the subject once a week for 1, 2, 3, 4, 5, or more weeks after the onset of symptoms.

In some embodiments, the treatment for fibrosis with an anti-alarmin binding molecule described herein results in a reduction in the ratio of lymphocytes relative to the total bronchoalveolar cells, a reduction in the ratio of neutrophils relative to the total bronchoalveolar cell, a reduction in total collagen, a reduction in fibronectin, and/or a reduction in one or more molecular pro-fibrotic mediators. In some embodiments, the molecular pro-fibrotic mediator is selected from transforming growth factor beta (TGF-β), connective tissue growth factor (CTGF), platelet-derived growth factor (PDGF), fibroblast growth factor (FGF), endothelin-1 (ET-1), IL-4, IL-5, IL-13, IL-21, MCP-1, MIP-1β, angiogenic factors (VEGF), TNF-α, peroxisome proliferator-activated receptors (PPARs), acute phase proteins (SAP), caspases, Angiotensin II, and endothelin (ET).

### Type 2 Inflammatory Diseases

Type 2 inflammatory diseases are a group of conditions that are characterized by an exaggerated immune response known as the type 2 immune response. The type 2 immune response is typically triggered by exposure to allergens. parasitic infections, or other environmental factors and is characterized by the activation of immune cells such as Th2 cells, eosinophils, mast cells, and basophils.

Examples of type 2 inflammatory diseases include asthma, viral exacerbations of allergic asthma, chronic rhinosinusitis with nasal polyps, allergic bronchopulmonary aspergillosis, atopic dermatitis, eosinophilic esophagitis, eosinophilic gastritis, eosinophilic gastroenteritis, eosinophilic colitis, allergic conjunctivitis, eosinophilia and food allergies. These conditions are characterized by inflammation and tissue damage, often resulting in chronic symptoms such as cough, wheezing, itching, and skin rashes.

Viral-induced type 2 inflammatory diseases are a group of conditions that are characterized by an immune response that is dominated by type 2 cytokines and the recruitment of type 2 immune cells, such as eosinophils, basophils, and T-helper 2 cells. These conditions can be triggered by a variety of viral infections, and they can affect various organ systems in the body, including the respiratory, gastrointestinal, and nervous systems. Non-limiting examples of viral-induced type 2 inflammatory diseases include asthma, chronic obstructive pulmonary disease (COPD). eosinophilic esophagitis (EoE), chronic rhinosinusitis with nasal polyps (CRSwNP), and viral encephalitis, acute respiratory syndrome (SARS), Middle East respiratory syndrome (MERS), and COVID-19.

Asthma is a chronic inflammatory disease of the airways that is characterized by bronchial hyperresponsiveness, wheezing, and shortness of breath. Viral infections, such as rhinovirus and respiratory syncytial virus (RSV), are a common trigger of asthma exacerbations, and they can induce a type 2 inflammatory response in the airways.

COPD is a chronic inflammatory lung disease that is characterized by the progressive obstruction of airflow. Viral infections, such as influenza and parainfluenza viruses, can trigger exacerbations of COPD, and they can induce a type 2 inflammatory response in the airways.

EoE is a chronic inflammatory disease of the esophagus that is characterized by eosinophilic infiltration of the esophageal tissue. Viral infections, such as herpes simplex virus (HSV), have been implicated in the development of EoE.

CRSwNP is a chronic inflammatory disease of the upper airways that is characterized by the presence of nasal polyps. Viral infections, such as rhinovirus and human metapneumovirus (hMPV), have been shown to induce a type 2 inflammatory response in the nasal tissue, which may contribute to the development of CRSwNP.

Viral encephalitis is an inflammation of the brain that is caused by viral infections, such as herpes simplex virus (HSV) and West Nile virus. Type 2 inflammatory cells, such as eosinophils, have been observed in the brains of some patients with viral encephalitis.

SARS is caused by a coronavirus, and it is characterized by fever, cough, shortness of breath, and respiratory failure. SARS can induce a type 2 inflammatory response in the lungs, which is characterized by the recruitment of eosinophils and the production of type 2 cytokines, such as interleukin-4 (IL-4) and interleukin-13 (IL-13). In addition to SARS, other coronaviruses, such as Middle East respiratory syndrome coronavirus (MERS-CoV) and severe acute respiratory syndrome coronavirus 2 (SARS-CoV-2), which causes COVID-19, can also induce a type 2 inflammatory response in the lungs. In COVID-19, the type 2 immune response is thought to play a role in the development of severe disease and lung damage.

Allergic exacerbations are a common feature of type 2 inflammatory diseases, and can occur when the immune system overreacts to an allergen, such as pollen, dust mites, or animal dander. In individuals with pre-existing type 2 inflammatory diseases, allergic exacerbations can lead to worsening of symptoms, increased inflammation, and a higher risk of disease exacerbation. Non-limiting examples of type 2 inflammatory diseases that are commonly associated with allergic exacerbations include asthma, allergic rhinitis, and atopic dermatitis. In asthma, exposure to allergens can trigger airway inflammation and bronchoconstriction, leading to symptoms such as cough, wheezing, and shortness of breath. In allergic rhinitis, exposure to allergens can trigger nasal congestion, runny nose, and sneezing. In atopic dermatitis, exposure to allergens can lead to skin inflammation and itching.

Environmental exacerbations are another common feature of type 2 inflammatory diseases, and can occur when individuals are exposed to environmental factors, such as air pollution, tobacco smoke, or certain chemicals, that trigger or worsen inflammation and exacerbate their symptoms. Non-limiting examples of type 2 inflammatory diseases that are commonly associated with environmental exacerbations include asthma, allergic rhinitis, and atopic dermatitis. In asthma, exposure to environmental pollutants, such as particulate matter, nitrogen oxides, and sulfur dioxide, can trigger airway inflammation and worsen symptoms. In allergic rhinitis, exposure to air pollution can exacerbate nasal congestion, runny nose, and sneezing. In atopic dermatitis, exposure to certain chemicals, such as detergents and solvents, can trigger skin inflammation and itching.

Drug-induced exacerbations are another potential trigger for type 2 inflammatory diseases. Some medications, such as non-steroidal anti-inflammatory drugs (NSAIDs), aspirin, beta-blockers, ACE inhibitors, and checkpoint inhibitors, can exacerbate symptoms in individuals with pre-existing type 2 inflammatory diseases, such as asthma and nasal polyps. NSAIDs and aspirin are known to trigger respiratory reactions in some individuals with asthma and nasal polyps, a condition known as aspirin-exacerbated respiratory disease (AERD). In AERD, exposure to NSAIDs and aspirin can cause nasal congestion, rhinorrhea, and wheezing, and can also exacerbate asthma symptoms. Beta-blockers, which are used to treat hypertension and other cardiovascular conditions, can also exacerbate asthma symptoms by blocking the activity of beta-2 adrenergic receptors in the airways, leading to bronchoconstriction and worsening of symptoms. ACE inhibitors are a class of medications commonly used to treat high blood pressure and heart failure. However, in some individuals, ACE inhibitors can induce a type 2 immune response and exacerbate symptoms of asthma and other type 2 inflammatory diseases.

In some embodiments, the present disclosure provides methods of preventing or treating a type 2 inflammatory disease in a subject in need thereof comprising administering an anti-alarmin binding molecule to the subject. In some embodiments, the anti-alarmin binding molecule is selected from an anti-IL25 antibody, an anti-IL-33 antibody, or an anti-TSLP antibody described herein.

In some embodiments, the anti-alarmin binding molecules are administered to the subject prior to the onset of symptoms in order to prevent one or more symptoms of a type 2 inflammatory disease. In some embodiments, the anti-alarmin binding molecules are administered to the subject after the onset of symptoms in order to treat one or more symptoms of a type 2 inflammatory disease. In some embodiments, the anti-alarmin binding molecules are administered to the subject 1, 2, 3, 4, 5, 6, 7, 8, 9, 10 or more times. In some embodiments, the anti-alarmin binding molecules are administered to the subject once a week for 1, 2, 3, 4, 5, or more weeks after the onset of symptoms.

In some embodiments, the treatment for a type 2 inflammatory disease with an anti-alarmin binding molecule results in a reduction in type 2 cytokine (e.g. IL 13, IL4 and IL5) expression. In some embodiments, the treatment results in a reduction in eotaxin and eosinophils. Eotaxin is a chemokine that plays a role in the recruitment of eosinophils, a type of white blood cell that is involved in type 2 inflammatory responses. Eotaxin is produced by a variety of cells, including epithelial cells, endothelial cells, and immune cells, in response to various stimuli, such as allergens, cytokines, and other inflammatory mediators. Eotaxin is particularly important in the pathogenesis of eosinophilic inflammatory diseases, such as asthma and eosinophilic esophagitis. In these diseases, eotaxin is produced in response to environmental allergens, and it attracts eosinophils to the site of inflammation. Eosinophils release inflammatory mediators, such as cytokines, proteases, and reactive oxygen species, which contribute to tissue damage and inflammation.

### Viral or Other Pathogen Infection

Respiratory viral infections are a common type of viral infection that affect the respiratory system, including the lungs, nose, and throat. Some common examples of respiratory viral infections include the common cold, influenza, respiratory syncytial virus (RSV), and COVID-19. Respiratory viral infections can cause a wide range of symptoms, including coughing, sneezing, sore throat, runny or stuffy nose, fever, and difficulty breathing. In severe cases, respiratory viral infections can lead to pneumonia, acute respiratory distress syndrome (ARDS), or other complications.

Airway epithelial cells are the primary site of respiratory viral infection and are critical to initiating anti-viral immunity. In the lungs, bronchial epithelial cells (BECs) induce an antiviral response through the production of type 1 interferon-β (IFN-β) and type III IFN-λ which in turn induce expression of IFN-stimulated genes (ISGs) that directly interfere with viral replication, enhance viral antigen presentation, and activate the adaptive immunity. Deficient/delayed type 1 and type III IFN production by RV-infected BECs from patients with asthma has been identified and this is thought to contribute to enhanced airway inflammation and bronchoconstriction and more severe disease. Without being bound by a theory, it is thought that an anti-alarmin binding molecule directly regulates BEC innate immunity during viral infection and inhibition of an alarmin (in addition to suppressing type 2 inflammation) increases interferon expression and reduces viral load.

Alarmins are also known to be involved in inflammatory responses to microbial infection and inflammatory symptoms associated with a microbial infection. Left unchecked, the microbial inflammation will reach the end stage inflammatory condition known as sepsis. As used herein, “microbial inflammation” refers to a condition associated with its cardinal signs such as redness, swelling, increase in temperature, pain, and impairment of organ function such as disordered respiration as a result of the epithelial injury with adjacent microvascular endothelial injury in the lungs (and other organs) due to a microbial infection such as a virus, bacteria, fungi, or parasite. That is, “Microbial inflammation” is a mechanism of disease caused by infection (“microbial insult”). Microbial inflammation evolves from innate immune response to an infection due to a microbe such as, for example, a virus, bacterium, fungus, or parasite. Thus, the microbial injury caused by microbial virulence factors is aggravated by the host-produced inflammatory mediators that impede the clearance of invading microbes and add insult to organ's injury. It is understood and herein contemplated that the microbial inflammation and its end stage, sepsis can result from any microbial insult elicited by known (or unknown) virulence factors and microbial antigens.

The innate and adaptive immune response to infecting pathogen (disease-causing microorganism) can include the burst in production of cytokines, chemokines, and proteolytic enzymes by granulocytes, monocytes, macrophages, dendritic cells, mast cells, innate lymphoid cells, T cells, B cells, NK cells, and NK T cells. Microbial inflammation can be localized to a specific organ- or can be systemic. Microbial inflammation can proceed in stages from acute to subacute and chronic with attendant tissue destruction and subsequent fibrosis. Left unchecked, the acute microbial inflammation can lead to sepsis and septic shock, the end stage of microbial inflammation.

It is understood that the pathogen can be a virus. Thus in one embodiment the pathogen can be selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus (including, but not limited to avian coronavirus (IBV), porcine epidemic diarrhea virus (PEDV), porcine respiratory coronavirus (PRCV), transmissible gastroenteritis virus (TGEV), feline coronavirus (FCoV), feline infectious peritonitis virus (FIPV), feline enteric coronavirus (FECV), canine coronavirus (CCoV), rabbit coronavirus (RaCoV), mouse hepatitis virus (MHV), rat coronavirus (RCoV), sialodacryadenitis virus of rats (SDAV), bovine coronavirus (BCoV), bovine enterovirus (BEV), porcine coronavirus HKU15 (PorCoV HKU15), Porcine epidemic diarrhea virus (PEDV), porcine hemagglutinating encephalomyelitis virus (HEV), turkey bluecomb coronavirus (TCoV), human coronavirus (HCoV)-229E, HCoV—OC43, HCoV—HKU1, HCoV-NL63, Severe Acute Respiratory Syndrome (SARS)-Coronavirus (CoV)(SARS-CoV), Severe Acute Respiratory Syndrome (SARS)-Coronavirus (CoV)-2 (SARS-CoV-2) (including, but not limited to the B1.351 variant, B.1.1.7 variant, and P.1 variant), or middle east respiratory syndrome (MERS) coronavirus (CoV) (MERS-CoV)), Influenza virus A. Influenza virus B, Measles virus, Polyomavirus. Human Papillomavirus, Respiratory syncytial virus, Adenovirus, Coxsackie virus, Chikungunya virus, Dengue virus, Mumps virus, Poliovirus, Rabies virus, Rous sarcoma virus, Reovirus, Yellow fever virus, Ebola virus, Marburg virus, Lassa fever virus. Eastem Equine Encephalitis virus, Japanese Encephalitis virus, St. Louis Encephalitis virus, Murray Valley fever virus, West Nile virus, Rift Valley fever virus, Rotavirus A. Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed are methods wherein the pathogen is a bacterium. The pathogen can be selected from the group of bacteria consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium chimaera, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriac, Shigella sonnei, Shigella flexneri, other Shigella species. Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella bumetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species, and Mycoplasma species. In one aspect the bacteria is not Bacillus anthracis.

Also disclosed are methods wherein the pathogen is a fungus selected from the group of fungi consisting of Candida albicans, Cryptococcus neoformans, Histoplasma capsulatum, Aspergillus fumigatus, Coccidiodes immitis, Paracoccidioides brasiliensis, Blastomyces dermitidis, Pneumocystis carinii, Penicillium mameffi, and Altemaria altemata.

Also disclosed are methods wherein the pathogen is a parasite selected from the group of parasitic organisms consisting of Toxoplasma gondii, Plasmodium falciparum, Plasmodium vivax, Plasmodium malariae, other Plasmodium species. Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica. Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and Entamoeba histolytica.

In some embodiments, the present disclosure provides methods of preventing or treating a pathogen infection in a subject in need thereof comprising administering an anti-alarmin binding molecule to the subject. In some embodiments, the anti-alarmin binding molecule is selected from an anti-IL25 antibody, an anti-IL-33 antibody, or an anti-TSLP antibody described herein.

In some embodiments, the anti-alarmin binding molecules are administered to the subject prior to the onset of symptoms in order to prevent one or more symptoms of a pathogen infection. In some embodiments, the anti-alarmin binding molecules are administered to the subject after the onset of symptoms in order to treat one or more symptoms of a pathogen infection. In some embodiments, the anti-alarmin binding molecules are administered to the subject 1, 2, 3, 4, 5, 6, 7, 8, 9, 10 or more times. In some embodiments, the anti-alarmin binding molecules are administered to the subject once a week for 1, 2, 3, 4, 5, or more weeks after the onset of symptoms.

### Formulations, Dosages, and Routes of Administration

In some embodiments, the anti-alarmin binding molecules described herein (e.g., anti-IL-25 antibodies, anti-IL-33 antibodies, and/or anti-TSLP antibodies) are administered to a subject in need thereof. Effective dosages and schedules for administering the anti-alarmin binding molecules described herein may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages forgiven classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above. In some embodiments, the therapeutically effective dose of an anti-alarmin binding molecule according to the present disclosure is about 5 mg/kg, about 10 mg/kg, about 20 mg/kg, about 30 mg/kg, about 40 mg/kg, or about 50 mg/kg. In some embodiments, the therapeutically effective dose of the anti-IL-25 antibody according to the present disclosure is about 5 mg/kg, about 10 mg/kg, about 20 mg/kg, about 30 mg/kg, about 40 mg/kg, or about 50 mg/kg.

As described above, the anti-alarmin binding molecules described herein (e.g., anti-IL-25 antibodies, anti-IL-33 antibodies, and/or anti-TSLP antibodies) are formulated as a composition comprising a pharmaceutically acceptable carrier. The compositions may be administered orally, parenterally (e.g., intravenously), by intramuscular injection, by intraperitoneal injection, by subcutaneous injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions described herein (e.g., anti-IL-25 antibodies, anti-IL-33 antibodies, and/or anti-TSLP antibodies) by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the compositions, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989): Bagshawe, et al., Br. J. Cancer, 58:700-703, (1988): Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80. (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

The compositions, including antibodies, can be used therapeutically in combination with a pharmaceutically acceptable carrier. Suitable carriers and their formulations are described in Remington: The Science and Practice of Pharmacy (19th ed.) ed. A. R. Gennaro, Mack Publishing Company, Easton, PA 1995. Typically. an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents. anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally. by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions. and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's. or fixed oils. Intravenous vehicles include fluid and nutrient replenishers, electrolyte replenishers (such as those based on Ringer's dextrose), and the like. Preservatives and other additives may also be present such as, for example, antimicrobials. anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

In some embodiments, the anti-alarmin binding molecules described herein (e.g., anti-IL-25 antibodies, anti-IL-33 antibodies, and/or anti-TSLP antibodies) are administered by conventional routes, including, but not limited to, intravenous injection, intravenous drip, subcutaneous injection, local injection, intramuscular injection, intratumoral injection, intraperitoneal injection (such as intraperitoneal injection)), intracranial injection, or intracavitary injection In some embodiments, the anti-alarmin binding molecules described herein are administered topically (including ophthalmically, vaginally, rectally, intranasally), orally, by inhalation, or parenterally.

In the context of the methods of treatment described herein, the anti-alarmin binding molecules may be administered as a monotherapy (i.e., as the only therapeutic agent) or in combination with one or more additional therapeutic agents.

The present disclosure includes compositions and therapeutic formulations comprising any of the anti-alarmin binding molecules described herein in combination with one or more additional therapeutically active components, and methods of treatment comprising administering such combinations to subjects in need thereof.

The anti-alarmin binding molecules of the present disclosure may be co-formulated with and/or administered in combination with, e.g., cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, IL-21, IL-23, IL-25, IL-26, or antagonists of their respective receptors.

The anti-alarmin binding molecules of the present disclosure may also be administered and/or co-formulated in combination with antivirals, antibiotics, analgesics, corticosteroids, steroids, oxygen, antioxidants, metal chelators, IFN-gamma, and/or NSAIDs.

The additional therapeutically active component(s) may be administered just prior to, concurrent with, or shortly after the administration of an anti-alarmin binding molecule of the present disclosure. The present disclosure includes pharmaceutical compositions in which an anti-alarmin binding molecule of the present disclosure is co-formulated with one or more of the additional therapeutically active component(s).

According to certain embodiments of the present disclosure, a single dose of an anti-alarmin binding molecule (or a pharmaceutical composition comprising a combination of an anti-alarmin binding molecule and any of the additional therapeutically active agents mentioned herein) may be administered to a subject.

According to some embodiments of the present disclosure, multiple doses of an anti-alarmin binding molecule (or a pharmaceutical composition comprising a combination of an anti-alarmin binding molecule and any of the additional therapeutically active agents mentioned herein) may be administered to a subject over a defined time course. The methods of the present disclosure comprise sequentially administering to a subject multiple doses of an anti-alarmin binding molecule of the present disclosure. As used herein, “sequentially administering” means that each dose of an anti-alarmin binding molecule is administered to the subject at a different point in time, e.g., on different days separated by a predetermined interval (e.g., hours, days, weeks or months). The present disclosure includes methods which comprise sequentially administering to the patient a single initial dose of an anti-alarmin binding molecule, followed by one or more secondary doses of the anti-alarmin binding molecule, and optionally followed by one or more tertiary doses of the anti-alarmin binding molecule.

The terms “initial dose,” “secondary doses,” and “tertiary doses,” refer to the temporal sequence of administration of the anti-alarmin binding molecule of the present disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of an anti-alarmin binding molecule, but generally may differ from one another in terms of frequency of administration. In certain embodiments, however, the amount of anti-alarmin binding molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of anti-alarmin binding molecule which is administered to a patient prior to the administration of the very next dose in the sequence with no intervening doses.

The methods according to this aspect of the disclosure may comprise administering to a patient any number of secondary and/or tertiary doses of an anti-alarmin binding molecule. For example, in certain embodiments, only a single secondary dose is administered to the patient. In other embodiments, two or more (e.g., 2, 3, 4, 5, 6, 7.8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose may be administered at the same frequency as the other secondary doses. For example, each secondary dose may be administered to the patient 1 to 2 weeks or 1 to 2 months after the immediately preceding dose. Similarly, in some embodiments involving multiple tertiary doses, each tertiary dose may be administered at the same frequency as the other tertiary doses. For example, each tertiary dose may be administered to the patient 2 to 12 weeks after the immediately preceding dose. In certain embodiments of the present disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of once a month, then the maintenance doses may be administered to the patient once every six weeks, once every two months, once every three months, etc.).

### Antibodies Generally

Antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The disclosed monoclonal antibodies can be made using any procedure which produces monoclonal antibodies. For example, disclosed monoclonal antibodies can be prepared using hybridoma methods, such as those described by Kohler and Milstein. Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, VHH, and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions. deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### Human Antibodies

The disclosed human antibodies can be prepared using any technique. The disclosed human antibodies can also be obtained from transgenic animals. For example, transgenic, mutant mice that are capable of producing a full repertoire of human antibodies. in response to immunization, have been described (see, e.g., Jakobovits et al., Proc. Nal. Acad. Sci. USA, 90:2551-255 (1993); Jakobovits et al., Nature, 362:255-258 (1993): Bruggermann et al., Year in Immunol., 7:33 (1993)). Specifically. the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### Humanized Antibodies

Antibody humanization techniques generally involve the use of recombinant DNA technology to manipulate the DNA sequence encoding one or more polypeptide chains of an antibody molecule. Accordingly. a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework ofa human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986). Reichmann et al., Nature, 332:323-327 (1988). and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988). Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### Protein Variants

As discussed herein there are numerous variants of the anti-alarmin binding molecules and IL-25 binding CDRs and heavy and light chain variable regions disclosed herein that are known and herein contemplated. In addition, to the known functional strain variants there are derivatives of the IL-25 binding molecules and IL-25 binding CDRs and heavy and light chain variable regions which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 3
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 4
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 4, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu: Asn, Gin; Ser, Thr: Lys, Arg; and Phe. Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:4 sets forth a particular sequence of an anti-IL25 heavy chain variable domain and SEQ ID NO: 8 sets forth a particular sequence of an anti-IL-25 light chain variable domain. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981). by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is understood that for this mutation all of the nucleic acid sequences that encode this particular derivative of any of SEQ ID NO: 4, SEQ ID NO: 8, SEQ ID NO 12, SEQ ID NO: 13 are also disclosed.

It is understood that there are numerous amino acid and peptide analogs which can be incorporated into the disclosed compositions. For example, there are numerous D amino acids or amino acids which have a different functional substituent then the amino acids shown in Table 3 and Table 4. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—, —CH═CH— (cis and trans), —COCH₂—, —CH(OH)CH₂—, and —CHH₂SO-(These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—, cis and trans): Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—): each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1. Anti-Fibrosis Effects of Anti-IL25 Antibodies In Vivo

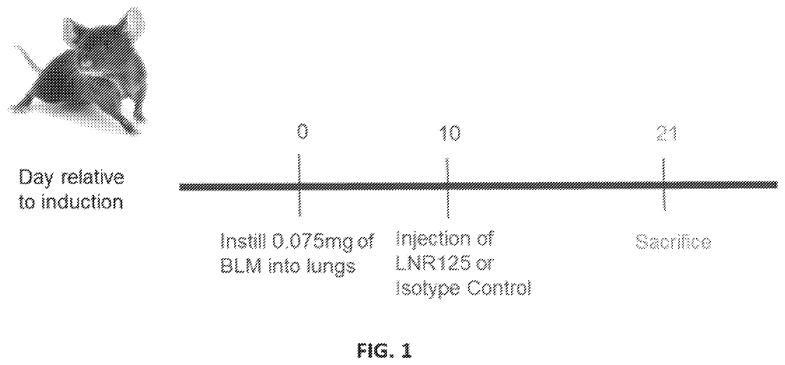

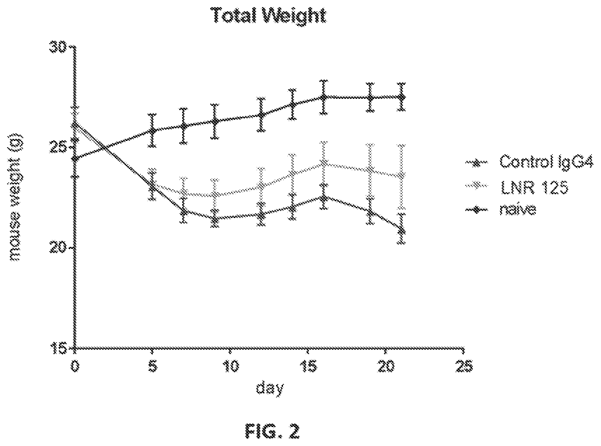

FIG. 1 illustrates the experiment protocol for evaluating the anti-fibrosis effects of anti-IL25 antibodies in bleomycin (BLM) treated mice. Male C57BL/6J mice 8 weeks of age were obtained from Jackson Laboratory (Bar Harbor, Maine). They were housed 4-5 to a cage in microisolator cages, fed standard rodent chow ad libitum, and had free access to water. After a standard acclimation period, testing began. On day 0, the mice were divided into 3 groups (n=6-8): naïve, isotype control (BLM+isotype control mAb), and LNR125 (BLM+LNR125). Bleomycin (BLM) was purchased from Caymen Chemical (Ann Arbor, MI) and was dissolved in PBS. All mice, except the naïve group, were anesthetized with isoflurane and given 3 mg/kg of Bleomycin mixed with 50 μl of PBS via oropharyngeal aspiration. On day 10, mice in the isotype and LNR125 groups were injected subcutaneously with 10 mg/kg of their respective mAb dissolved in PBS. On day 21, mice were sacrificed via isoflurane overdose and cervical dislocation. Body weights were recorded every 2-3 days. FIG. 2 shows the body weights of mice in the three groups. LNR125-treated mice had less weight loss compared to the isotype control group.

The tracheas were dissected and transected just distal to the larynx and lungs were dissected out and weighed. The trachea was cannulated with a 24 gauge catheter and then lavaged 2 times with 500 μl of PBS containing EDTA and a protease inhibitor. Bronchoalveolar lavage fluid (BALF) was centrifuged at 4 degrees Celsius at 500 g for 5 minutes. The supernatant was stored at −80 degrees Celsius. Total cell counts of the remaining pellet were performed on a hemocytometer and differential cell counts were obtained by smearing a portion of the BALF pellet on a microscope slide, staining with Diff Quik (Romanowsky stain variant), and counting 200 cells at 40×. One lung lobe was snap frozen in liquid nitrogen. At the time of analysis, lung samples were weighed, placed into 6M HCl (100 mg/ml), and incubated at 95 degrees Celsius for 20 hours. The samples were then centrifuged and the supematants used for collagen quantification using the total collagen assay kit (QuickZyme Biosciences, The Netherlands) per manufacturer's instructions. Another lung lobe was placed in 10% buffered formalin. After fixation in formalin, full thickness lung samples were embedded in paraffin, sliced, and stained with H&E and Masson's Trichrome to evaluate inflammatory cells/morphology and collagen deposition, respectively. Fibrosis scores were assessed by observing 10 fields per sample. Scores of 0-8, according to the Ashcroft scale (0=normal, 8=total fibrosis), were assigned and averaged. Grading was based on alveolar septae thickening, loss of normal architecture, and collagen deposition. Microscopic images were also captured digitally and analyzed with ImageJ software to determine area of collagen staining. Immunohistochemistry was also performed on histology slides to assess the levels of IL-25 within the tissue. One set of slides were stained with biotinylated LNR125 and another set where stained with the receptor (IL-17RB) antibody, LNR 226, both at a 1:100 dilution. Streptavidin HRP was used as a secondary and Diva substrate was used as the capture.

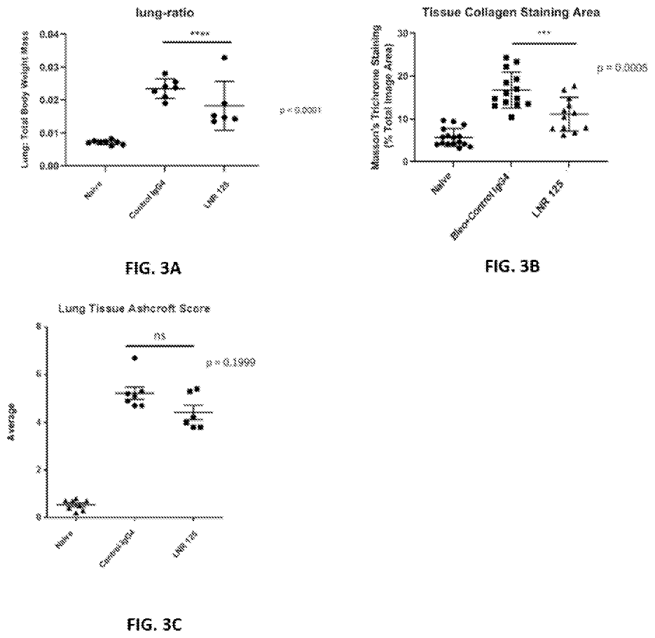

Though not all scores were significant, mice treated with LNR125 10 days after administration of BLM had less evidence of fibrosis based on lung to body weight ratio (FIG. 3A), tissue collagen staining area (FIG. 3B), and Ashcroft score (FIG. 3C). FIG. 3A shows that lung index (a ration of lung weight to body weight) was significantly lower by about 22% in the LNR125 group compared to the isotype control group. FIG. 3B shows the measurements of microscopic lung collagen staining area using ImageJ software. The data show that LNR125 treated mice had a reduction in lung collagen content by about 27% compared to the isotype control. Fibrosis scores were assessed by observing 10 fields per sample. Scores of0-8, according to the Ashcroft scale (0=normal, 8=total fibrosis), were assigned and averaged. Grading was based on alveolar septae thickening, loss of normal architecture, and collagen deposition. Microscopic images were also captured digitally and analyzed with ImageJ software to determine area of collagen staining. FIG. 3C shows that fibrosis scoring was decreased in the LNR125 treated group compared to the isotype control group. The reduction in fibrosis score in the LNRI25 treated group is about 19% compared to the isotype control group.

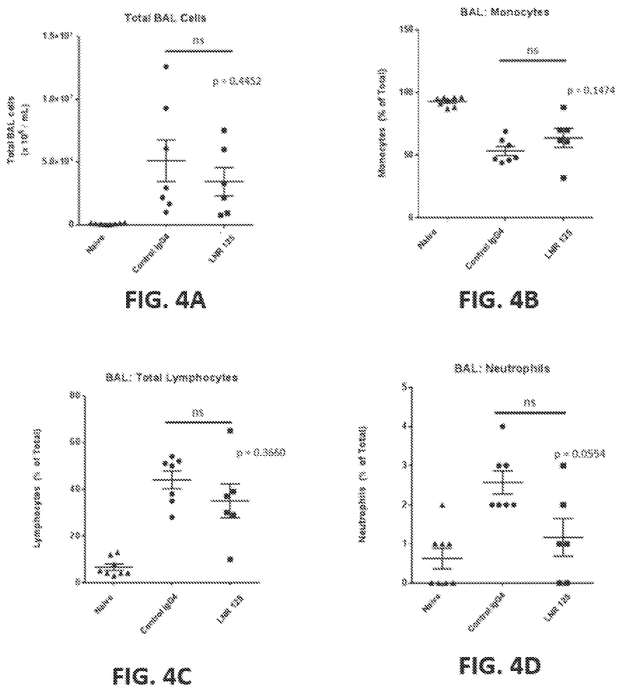

Bronchoalveolar lavage fluid was analyzed on Day 21 to ascertain the types of inflammatory cells present within the alveolar spaces of the lungs. FIGS. 4A-4D shows the cell counts of total bronchoalveolar cells (FIG. 4A). monocytes in bronchoalveolar cells (FIG. 4B), total lymphocytes in bronchoalveolar cells (FIG. 4C), and neutrophils in bronchoalveolar cells (FIG. 4D). Though the total cell count in the BALF was not different between the pulmonary fibrosis groups, the LNR125 treated group did have an overall better profile of differential cells within the BALF as compared to the isotype control group. (FIG. 4A). The percentages of normal alveolar macrophages were higher in the LNR125 group (FIG. 4B) and the percentages of lymphocytes and neutrophils were lower (FIGS. 4C and 4D) in the LNR125 group as compared to the group of mice that received the isotype control treatment. The results show a significant decrease in neutrophils (about 54%) in BALF in mice of the LNR125 group compared to the isotype control group (FIG. 4D).

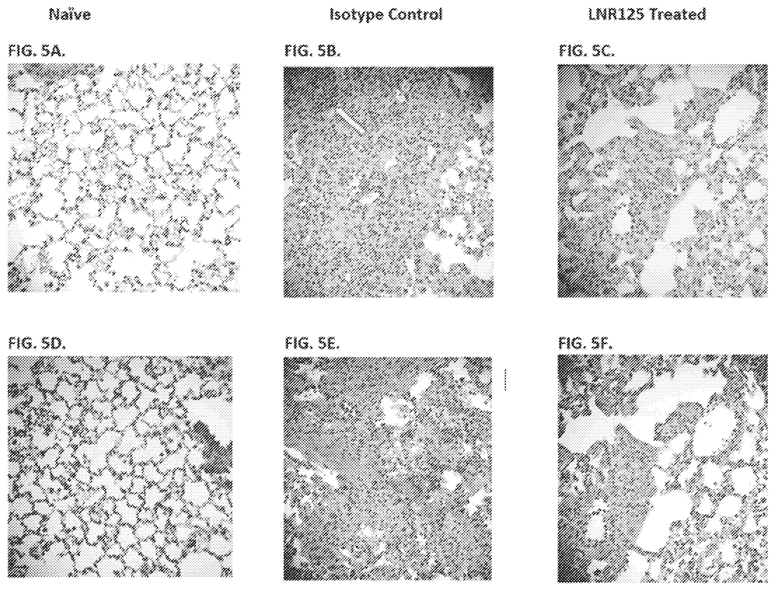

Lung samples that were fixed in formalin and embedded in paraffin were stained with H&E (FIGS. 5A-5C) and Masson's Trichrome (FIGS. 5D-5E) to visually ascertain the level of inflammation and fibrosis, respectively. Mice in the isotype control group had more clusters of lymphocytes (indicating inflammation) (FIG. 5B) and more areas of fibrosis (FIG. 5E) than the mice in the LNR125 treated group (FIGS. 5C and 5F). The data show that LNR125 treated mice had a reduction in lung collagen content by about 27% compared to the isotype control.

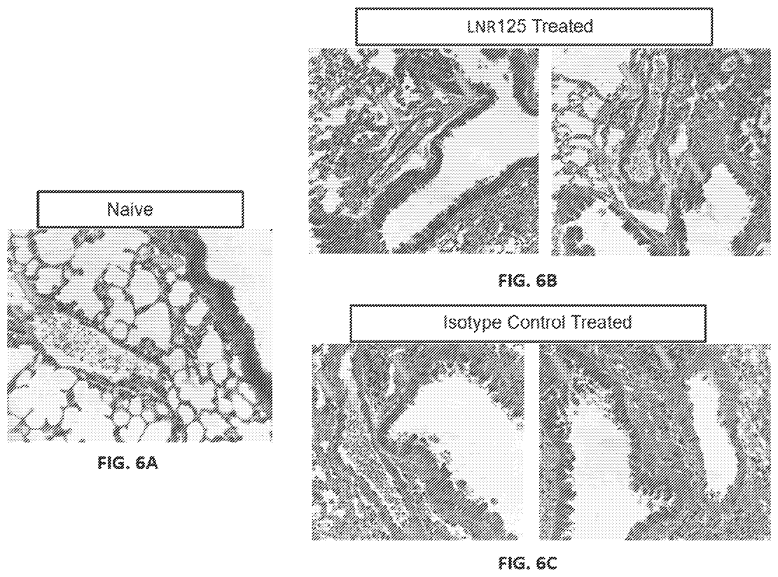

FIGS. 6A-6C shows the smoothness and thinness of the arteriole wall of the naïve and LNR125 treated mice versus the thicker arteriole wall of the isotype control group, in which fibrotic changes can be observed.

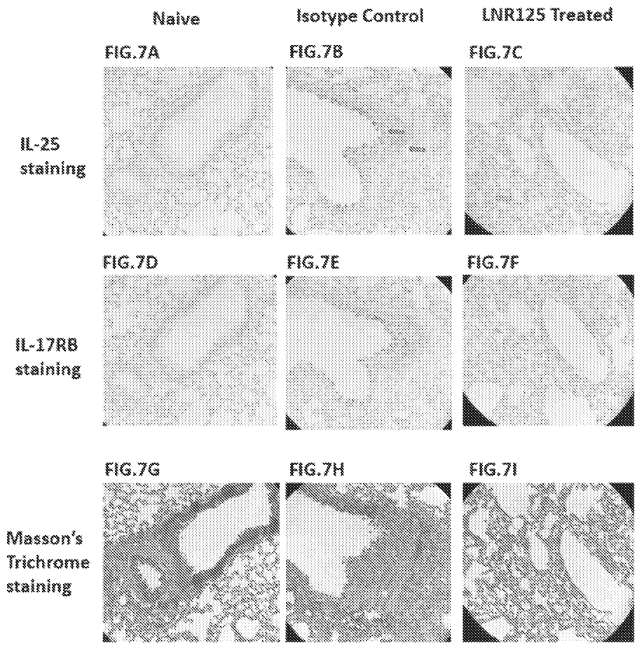

In a separate experiment with another group of mice under the same conditions, formalin fixed paraffin embedded lung samples were analyzed via immunohistochemistry to determine the amount of IL-25 present in this model of pulmonary fibrosis. Tissue samples were stained with either LNR125 (anti-IL-25 mAb) or with LNR226 (anti IL-17RB mAb), which is the receptor for IL-25. FIGS. 7A-71 show that naïve mice had little to no staining for IL-25 (FIG. 7A) whereas the tissue samples from the mice treated bleomycin (FIG. 7B) had positive stain uptake. The mice in the LNR125 treated group had less staining for both IL-25 (FIG. 7C) and IL-17RB (FIG. 7F) than the mice treated with the isotype control. The results of this study indicate that LNR125 treatment reduced inflammation and fibrosis relative to the isotype control.

### Example 2. Anti-IL25 Antibodies Block Proliferation of Fibroblast Ex Vivo

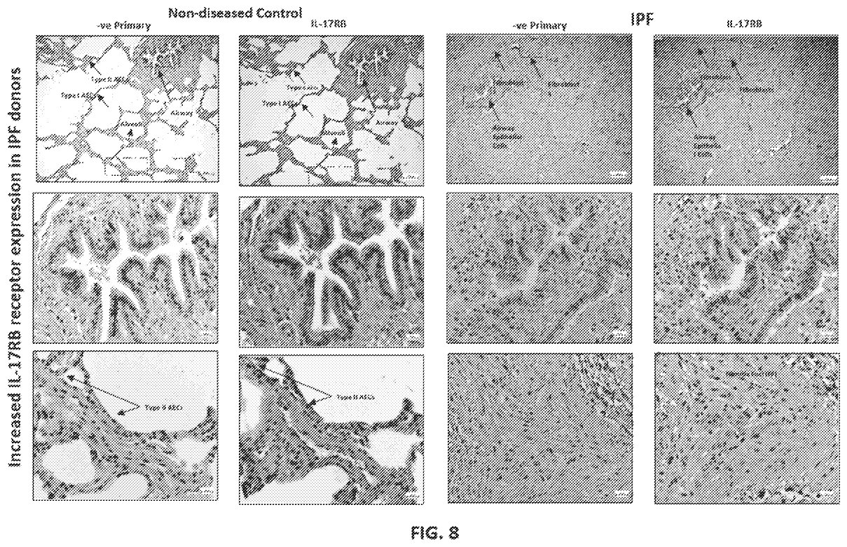

Human tissue array of lung samples from patients diagnosed with idiopathic pulmonary fibrosis (IPF) were obtained and prepared for IHC to detect the expression of IL-17 RB. FIG. 8 shows an enhanced expression of IL-17RB in the patient with IPF compared to no-disease control.

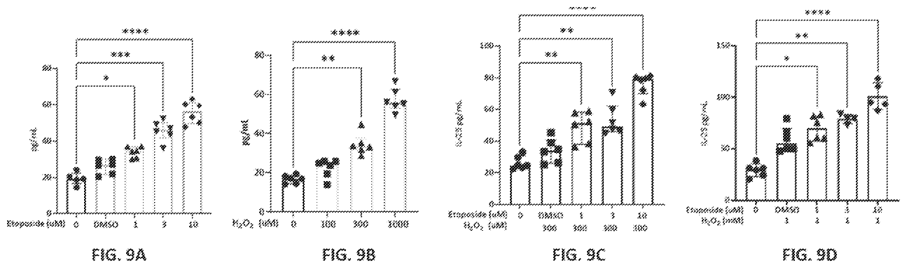

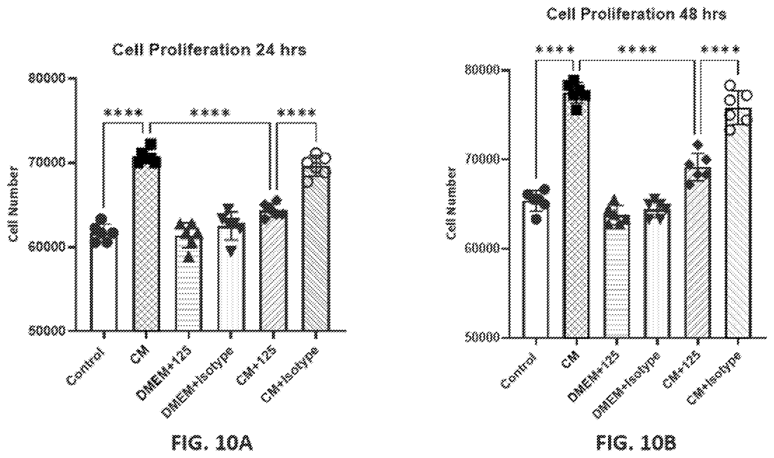

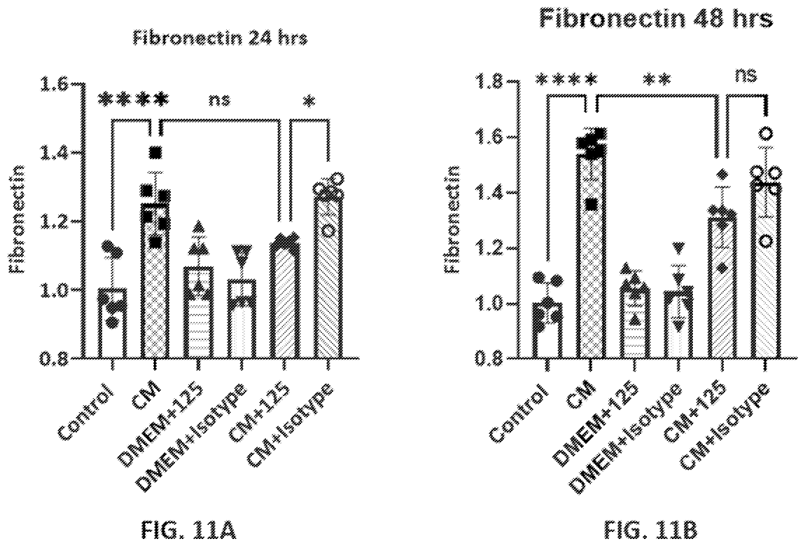

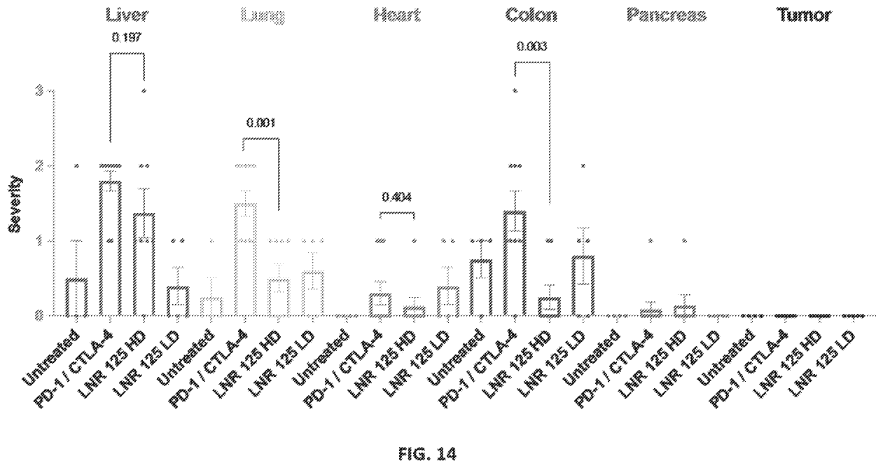

FIGS. 9A-9D show that etoposide/H2O2 was used to stress alveolar epithelial cells (A549 cells) to express high level IL-25 protein. The study showed that blockade of IL-25 with LNR125 reduced cell proliferation of MRC-5 fibroblasts stimulated with conditioned medium (CM) from H202/etoposide stressed alveolar epithelial cells (A549 cells) for 24 (FIG. 10A) and for 48 (FIG. 10B) hours, respectively. Moreover, blockade of IL-25 with LNR125 prevented CM induced expression of pro-fibrotic mediators—Fibronectin by MRC-5 fibroblasts after 24 (FIG. 11A) and 48 (FIG. 11B) hours of stimulation, respectively. Additionally, blockade of IL-25 reduces CM-induced collagen I & III gene expression in MRC-5 fibroblasts after 24 hours (FIGS. 14A and 14C) and 48 hours (FIGS. 14B and 14D) of stimulation, respectively.

These results indicate that LNR125 inhibits fibroblast proliferation in fetal lung fibroblasts (MRC-5) as well as blocks expression of pro-fibrotic mediators. Treating fibroblasts with CM from etoposide/H2O2 stressed alveolar epithelial cells (A549 cells) induces fibroblast proliferation and neutralizing IL25 in CM with LNR125 reverses this proliferative effect.

### Example 3. Anti-irAE Effects of Anti-IL-25 Antibodies in a Mouse Model of Checkpoint Inhibitor

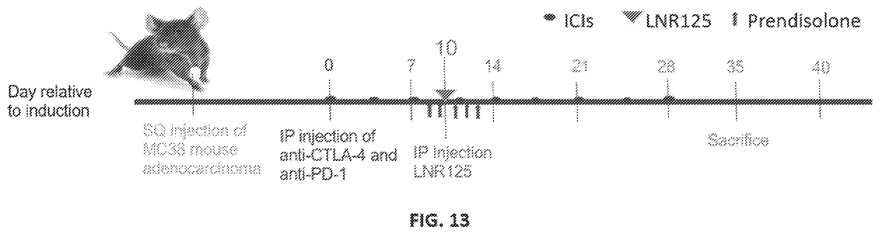

Experiments are performed to determine the efficacy of the anti-IL-25 antibodies described herein in a mouse model of checkpoint inhibitor induced pneumonitis (CIP). An experimental protocol is provided in FIG. 13.

Prior to study initiation baseline blood samples are collected from all mice for basic metabolic panels and anti-nuclear anti-body titers. Following initial blood collection, MC38 mouse colon adenocarcinoma cells are injected SQ into 32, 6-9 week old B6/lpr mice. When tumor volume reaches 100 mm³ mice are injected intraperitoneally with immune checkpoint inhibitors (ICI) (anti-CTLA-4 and anti-PD-1) beginning on day 0. ICI injections continue twice a week at 200 and 100 μg respectively. On week 2, prednisolone is given orally for 5 days at 1 mg/kg. On day 10. mice in the isotype control and treatment groups receive an injection of LNR 125 IP. CFA is injected IP on day 35. Body weights are recorded every 2-3 days and mice are monitored for tumor growth. At day 40, mice are euthanized, blood collected and liver, lung (BALF and lung tissue), pancreas and colon tissues samples taken for histopathology, cell counts and differentials and cytokine analysis.

Experimental groups for these experiments are as follows:
a) Naïve
b) Anti-PD-1+anti-CTLA-4 induced (PD-CT)
c) Anti-PD-1+anti-CTLA-4 induced+LNR125 (10 mg/kg) (PD-CT+LNR125) treatment
d) Anti-PD-1+anti-CTLA-4 induced+LNR125 (20 mg/kg) (PD-CT+LNR125) treatment
e) Anti-PD-1+anti-CTLA-4 induced+Prednisone (PD-CT+steroid) treatment.

ICIs are associated with a spectrum of inflammatory side effects termed immune-related adverse events (IrAEs). Immune-related pneumonitis, which is defined as a focal or diffuse inflammation of the lung parenchyma, is one of the few potentially life-threatening IrAEs. To characterize IrAEs in various organs and tissues, tissues from liver, lung, heart, colon, pancreas and tumor were extracted, fixed in 10% formalin and processed for H&E and anti-CD19 (D4V4B), CD8a (D4W2Z), and CD4 (D7D2Z) immunohistochemistry (IHC). Goat anti IgG H+L biotinylated antibody was used to detect rat IgG anti-mouse PD-1. Slides were scanned using Leica SCN400 and visualized with Aperio ImageScope. Two blinded pathologists assisted with grading of immune infiltration. Two slides were made from each organ. Five random high-power filed images were scored and averaged.

FIG. 14 shows that administration of anti-PD-1 and anti-CTLA-4 ICI resulted in significantly increased leukocytes infiltration in the liver, lungs, heart, colon, pancreas, and tumor tissues. A single dose of LNR 125 reduced leukocytes infiltration, especially in the lung and colon, where the LNR 125-mediated reduction reached a significance. The results indicate that a single dose of LNR 125 was sufficient to prevent the onset of pneumonitis in animals in a dose-dependent fashion.

### Example 4. A Prophetic Phase 1b/2 Study to Evaluate the Administration of an Anti-Alarmin Binding Molecule with Concomitant Pembrolizumab Treatment for Prevention or Treatment of Pneumonitis

### Brief Summary

A Phase 1b/2 study is conducted that will evaluate the efficacy of treatment with an anti-alarmin binding molecule selected from an anti-IL25 antibody, an anti-IL-33 antibody, and an anti-TSLP antibody before, during, or after concomitant immune checkpoint inhibitor (ICI) treatment using the PD-1 inhibitor pembrolizumab for the prevention or treatment of pneumonitis. This study will consist of two parts: a dose search part of the study (Phase 1b) and the dose expansion part of the study (Phase 2).

### Study Design

This two part study will enroll patients with advanced malignancies who will be treated with concomitant pembrolizumab ICI combination therapy. Phase 1b will evaluate a range of anti-alarmin antibody doses to test for safety and efficacy. The safe, recommend dose identified in Phase 1b will then be used in Phase 2 to further assess anti-pneumonitis activities of the anti-alarmin antibody over placebo.

### Dosing Scheme

Phase 1b: Patients will be divided among six dosages: Dose 1, 5 mg/kg anti-alarmin antibody administered once; Dose 2, 10 mg/kg anti-alarmin antibody administered once; Dose 3, 20 mg/kg anti-alarmin antibody administered once; Dose 4, 30 mg/kg anti-alarmin antibody administered once; Dose 5, 40 mg/kg anti-alarmin antibody administered once; Dose 6, 50 mg/kg anti-alarmin antibody administered once.

Phase 2: Using dose identified in Phase 1b, a new patient cohort will be divided among three dosing groups:
1. Group 1: Administration of the anti-alarmin antibody or placebo to patients prior to the first administration of concomitant pembrolizumab ICI treatment.
2. Group 2: Administration of the anti-alarmin antibody or placebo to patients concurrently with the first administration of concomitant pembrolizumab ICI treatment.
3. Group 3: Administration of the anti-alarmin antibody or placebo to patients within 1-10 days after the first administration of concomitant pembrolizumab ICI treatment.

The expected full duration of each patient's participation in this study is 24 months.

### Example 5. A Prophetic Phase 1b/2 Study to Evaluate the Administration of Anti-Alarmin Antibody with Concomitant Ipilimumab Treatment for Prevention or Treatment of Pneumonitis

### Brief Summary

A Phase 1b/2 study is conducted that will evaluate the efficacy of treatment with an anti-alarmin binding molecule selected from an anti-IL25 antibody, an anti-IL-33 antibody, and an anti-TSLP antibody before, during, or after concomitant immune checkpoint inhibitor (ICI) treatment using the CTLA-4 inhibitor ipilimumab for the prevention or treatment of pneumonitis. This study will consist of two parts: a dose search part of the study (Phase 1b) and the dose expansion part of the study (Phase 2).

### Study Design

This two part study will enroll patients with advanced malignancies who will be treated with concomitant ipilimumab ICI combination therapy. Phase 1b will evaluate a range of anti-alarmin antibody doses to test for safety and efficacy. The safe, recommend dose identified in Phase 1b will then be used in Phase 2 to further assess anti-pneumonitis activities of the anti-alarmin antibody over placebo.

### Dosing Scheme

Phase 1b: Patients will be divided among six dosages: Dose 1, 5 mg/kg anti-alarmin antibody administered once; Dose 2, 10 mg/kg anti-alarmin antibody administered once; Dose 3, 20 mg/kg anti-alarmin antibody administered once; Dose 4, 30 mg/kg anti-alarmin antibody administered once; Dose 5, 40 mg/kg anti-alarmin antibody administered once; Dose 6, 50 mg/kg anti-alarmin antibody administered once.

Phase 2: Using dose identified in Phase 1b, a new patient cohort will be divided among three dosing groups:
1. Group 1: Administration of the anti-alarmin antibody or placebo to patients prior to the first administration of concomitant ipilimumab ICI treatment.
2. Group 2: Administration of the anti-alarmin antibody or placebo to patients concurrently with the first administration of concomitant ipilimumab ICI treatment.
3. Group 3: Administration of the anti-alarmin antibody or placebo to patients within 1-10 days after the first administration of concomitant ipilimumab ICI treatment.

The expected full duration of each patient's participation in this study is 24 months.

### Example 6. A Prophetic Phase 1b/2 Study to Evaluate the Administration of Anti-Alarmin Antibody with Concomitant Pembrolizumab and Ipilimumab Treatment for Prevention or Treatment of Pneumonitis

### Brief Summary

A Phase 1b/2 study is conducted that will evaluate the efficacy of treatment with an anti-alarmin binding molecule selected from an anti-IL25 antibody, an anti-IL-33 antibody, and an anti-TSLP antibody before, during, or after concomitant immune checkpoint inhibitor (ICI) treatment using the PD-1 inhibitor pembrolizumab and CTLA-4 inhibitor ipilimumab for the prevention or treatment of pneumonitis. This study will consist of two parts: a dose search part of the study (Phase 1b) and the dose expansion part of the study (Phase 2).

### Study Design

This two part study will enroll patients with advanced malignancies who will be treated with concomitant pembrolizumab and ipilimumab ICI combination therapy. Phase 1b will evaluate a range of anti-alarmin antibody doses to test for safety and efficacy. The safe, recommend dose identified in Phase 1b will then be used in Phase 2 to further assess anti-pneumonitis activities of the anti-alarmin antibody over placebo.

### Dosing Scheme

Phase 1b: Patients will be divided among six dosages: Dose 1, 5 mg/kg anti-alarmin antibody administered once: Dose 2, 10 mg/kg anti-alarmin antibody administered once; Dose 3, 20 mg/kg anti-alarmin antibody administered once; Dose 4, 30 mg/kg anti-alarmin antibody administered once: Dose 5, 40 mg/kg anti-alarmin antibody administered once: Dose 6, 50 mg/kg anti-alarmin antibody administered once.

Phase 2: Using dose identified in Phase 1b, a new patient cohort will be divided among three dosing groups:
1. Group 1: Administration of the anti-alarmin antibody or placebo to patients prior to the first administration of concomitant pembrolizumab and ipilimumab ICI treatment.
2. Group 2: Administration of the anti-alarmin antibody or placebo to patients concurrently with the first administration of concomitant pembrolizumab and ipilimumab ICI treatment.
3. Group 3: Administration of the anti-alarmin antibody or placebo to patients within 1-10 days after the first administration of concomitant pembrolizumab and ipilimumab ICI treatment.

The expected full duration of each patient's participation in this study is 24 months.

1. A method of preventing or treating pneumonitis in a subject in need thereof comprising administering an anti-alarmin binding molecule to the subject.

2. The method of claim 1, wherein the pneumonitis is the result of interstitial lung disease, viral infection, autoimmune disease, allergy, inhalation of occupational debris, dusts, fibers, fumes or vapors, inhalation of chemicals or molds, sepsis, adverse reaction to medications, aspirin overdose, hypersensitivity to environmental antigens, overexposure to chlorine, exposure to herbicides, fluorocarbons, radiation, chemotherapy, and/or treatment with one or more immune checkpoint inhibitors.

3. The method of claim 1, wherein the subject is at risk for developing pneumonitis.

4. The method of claim 3, wherein the subject has a disease condition selected from chronic obstructive pulmonary disease (COPD), ulcerative colitis (UC), lung cancer, gastrointestinal (GI) cancer, pulmonary fibrosis, atopic dermatitis, asthma, or eosinophilic esophagitis (EOE).

5. The method of claim 2, wherein the one or more checkpoint inhibitors are selected from an anti-programmed death receptor-1(PD1) molecule, an anti-programmed death ligand 1 (PD-L1) molecule, an anti-cytotoxic T-lymphocyte associated protein 4 (CTLA4) molecule, an anti-Lymphocyte-activation gene 3 (LAG3) molecule, an anti-T-cell immunoreceptor with Ig and ITIM domains (TIGIT) molecule, an anti-T-cell immunoglobulin and mucin domain-containing protein 3 (TIM-3) molecule, an anti-V-domain Ig suppressor of T cell activation (VISTA) molecule, an anti-B and T lymphocyte attenuator (BTLA) molecule, an anti-Sialic acid-binding Ig-like lectin 15 (Siglec-15) molecule, and an anti-CD96 molecule.

6. The method of claim 5, wherein the anti-PD1 molecule comprises an anti-PD1 antibody selected from a group consisting of pembrolizumab, nivolumab, cemiplimab, dostarlimab, spartalizumab, camrelizumab, sintilimab, tislelizumab, toripalimab, and retifanlimab.

7. The method of claim 5, wherein the anti-CTLA4 molecule comprises an anti-CTLA4 antibody selected from a group consisting of ipilimumab, tremelimumab, BMS-986249, quavonlimab, and AGEN1884.

8. The method of claim 5, wherein the anti-PD-L1 molecule comprises an anti-PD-L1 antibody selected from a group consisting of atezolizumab, avelumab, and durvalumab.

9. The method of claim 2, wherein the treatment with one or more checkpoint inhibitors comprises treatment with two checkpoint inhibitors.

10. The method of claim 9, wherein the two checkpoint inhibitors comprise an anti-PD1 antibody and an anti-CTLA4 antibody.

11. The method of claim 2, wherein the treatment with one or more checkpoint inhibitors comprises a cell therapy comprising CAT-T cells expressing one or more checkpoint inhibitors.

12. The method of claim 2, wherein the treatment with one or more checkpoint inhibitors comprises a cell therapy comprising allogeneic T cells expressing one or more checkpoint inhibitors.

13. The method of claim 2, wherein the treatment with one or more checkpoint inhibitors comprises a gene therapy comprising viral vectors expressing one or more checkpoint inhibitors.

14. The method of claim 2, wherein the treatment with one or more checkpoint inhibitors comprises one or more checkpoint inhibitors conjugated to a therapeutic moiety.

15. The method of claim 14, wherein the therapeutic moiety comprises a cytotoxic agent, a therapeutic agent, a radioisotope, an ultrasound sensitizer, or an exosome secretion inhibitor.

16. The method of claim 15, wherein the cytotoxic agent comprises taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof.

17. The method of claim 15, wherein the therapeutic agent comprises an antimetabolite, an alkylating agent, an anthracycline, an antibiotic, and an anti-mitotic agent.

18. The method of claim 17, wherein the antimetabolite comprises methotrexate, 6-mercaptopurine, 6-thioguanine, cytarabine, 5-fluorouracil dacarbazine.

19. The method of claim 17, wherein the alkylating agent comprises mechlorethamine, thioepa chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin.

20. The method of claim 17, wherein the anthracycline comprises daunorubicin and doxorubicin.

21. The method of claim 17, wherein the antibiotic comprises dactinomycin, bleomycin, mithramycin, and anthramycin (AMC).

22. The method of claim 17, wherein the anti-mitotic agent vincristine and vinblastine.

23. The method of claim 15, wherein the radioisotope is radioactive iodine.

24. The method of claim 15, wherein the ultrasound sensitizer comprises porphyrins, porphyrin isomers and expanded porphyrins.

25. The method of claim 15, wherein the exosome secretion inhibitor comprises Manumycin A, GW4869, cannabidiol and endothelin receptor antagonists.

26. The method of any one of claims 1 to 25, wherein the anti-alarmin binding molecule is administered after the onset of pneumonitis symptoms.

27. The method of any one of claims 1 to 25, wherein the anti-alarmin binding molecule is administered prior to the onset of pneumonitis symptoms.

28. The method of any one of claims 1 to 25, wherein the anti-alarmin binding molecule is administered with the onset of the treatment with one or more immune checkpoint inhibitors.

29. The method of any one of claims 1 to 28, wherein the anti-alarmin binding molecule is administered as a single dose.

30. The method of any one of claims 1 to 28, wherein the anti-alarmin binding molecule is administered as multiple doses.

31. A method of preventing or treating checkpoint inhibitor-induced pneumonitis in a subject in need thereof comprising administering an anti-alarmin binding molecule to the subject.

32. The method of claim 31, wherein the subject has previously received, is receiving or will receive treatment with one or more checkpoint inhibitors.

33. The method of claim 32, wherein the anti-alarmin binding molecule is administered at least 1, 2, 3, 4, 5, 6, 7, 8, 9, or 10 days after the administration of one or more checkpoint inhibitors.

34. The method of claim 32, wherein the anti-alarmin binding molecule is administered concurrently with one or more checkpoint inhibitors.

35. The method of claim 32, wherein the anti-alarmin binding molecule is administered prior to the administration of one or more checkpoint inhibitors.

36. The method of claim 35, wherein the anti-alarmin binding molecule is administered at least 1, 2, 3, 4, 5, 6, 7, 8, 9, or 10 days prior to the administration of the one or more checkpoint inhibitors.

37. The method of any one of claims 31 to 36, wherein the one or more checkpoint inhibitors are selected from an anti-programmed death receptor-1(PD1) molecule, an anti-programmed death ligand 1 (PD-L1) molecule, an anti-cytotoxic T-lymphocyte associated protein 4 (CTLA4) molecule, an anti-Lymphocyte-activation gene 3 (LAG3) molecule, an anti-T-cell immunoreceptor with Ig and ITIM domains (TIGIT) molecule, an anti-T-cell immunoglobulin and mucin domain-containing protein 3 (TIM-3) molecule, an anti-V-domain Ig suppressor of T cell activation (VISTA) molecule, an anti-B and T lymphocyte attenuator (BTLA) molecule, an anti-Sialic acid-binding Ig-like lectin 15 (Siglec-15) molecule, and an anti-CD96 molecule.

38. The method of claim 37, wherein the anti-PD1 molecule comprises an anti-PD1 antibody selected from a group consisting of pembrolizumab, nivolumab, cemiplimab, dostarlimab, spartalizumab, camrelizumab, sintilimab, tislelizumab, toripalimab, and retifanlimab.

39. The method of claim 37, wherein the anti-CTLA4 molecule comprises an anti-CTLA4 antibody selected from a group consisting of ipilimumab, tremelimumab, BMS-986249, quavonlimab, and AGEN1884.

40. The method of claim 37 wherein the anti-PD-L1 molecule comprises an anti-PD-L1 antibody selected from a group consisting of atezolizumab, avelumab, and durvalumab.

41. The method of any one of claims 31 to 40, wherein the treatment with one or more checkpoint inhibitors comprises treatment with two checkpoint inhibitors.

42. The method of claim 41, wherein the two checkpoint inhibitors comprise an anti-PD1 antibody and an anti-CTLA4 antibody.

43. The method of claim 32, wherein the treatment with one or more checkpoint inhibitors comprises a cell therapy comprising CAT-T cells expressing one or more checkpoint inhibitors.

44. The method of claim 32, wherein the treatment with one or more checkpoint inhibitors comprises a cell therapy comprising allogeneic T cells expressing one or more checkpoint inhibitors.

45. The method of claim 32, wherein the treatment with one or more checkpoint inhibitors comprises a gene therapy comprising viral vectors expressing one or more checkpoint inhibitors.

46. The method of claim 32, wherein the treatment with one or more checkpoint inhibitors comprises one or more checkpoint inhibitors conjugated to a therapeutic moiety.

47. The method of claim 46, wherein the therapeutic moiety comprises a cytotoxic agent, a therapeutic agent, a radioisotope, an ultrasound sensitizer, or an exosome secretion inhibitor.

48. The method of claim 47, wherein the cytotoxic agent comprises taxol, cytochalasin B, gramicidin D, ethidium bromide, emetine, mitomycin, etoposide, tenoposide, vincristine, vinblastine, colehicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof.

49. The method of claim 47, wherein the therapeutic agent comprises an antimetabolite, an alkylating agent, an anthracycline, an antibiotic, and an anti-mitotic agent.

50. The method of claim 49, wherein the antimetabolite comprises methotrexate, 6-mercaptopurine, 6-thioguanine, cytarabine, 5-fluorouracil dacarbazine.

51. The method of claim 49, wherein the alkylating agent comprises mechlorethamine, thioepa chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin.

52. The method of claim 49, wherein the anthracycline comprises daunorubicin and doxorubicin.

53. The method of claim 49, wherein the antibiotic comprises dactinomycin, bleomycin, mithramycin, and anthramycin (AMC).

54. The method of claim 49, wherein the anti-mitotic agent vincristine and vinblastine.

55. The method of claim 47, wherein the radioisotope is radioactive iodine.

56. The method of claim 47, wherein the ultrasound sensitizer comprises porphyrins, porphyrin isomers and expanded porphyrins.

57. The method of claim 47, wherein the exosome secretion inhibitor comprises Manumycin A, GW4869, cannabidiol and endothelin receptor antagonists.

58. The method of any one of claims 31 to 57, wherein the anti-alarmin binding molecule is administered as a single dose.

59. The method of any one of claims 31 to 57, wherein the anti-alarmin binding molecule is administered as multiple doses.

60. A method of preventing or treating fibrosis in a subject in need thereof comprising administering an anti-alarmin binding molecule to the subject.

61. The method of claim 60, wherein the fibrosis is pulmonary fibrosis, liver fibrosis, cardiac fibrosis, renal fibrosis, skin fibrosis, gastrointestinal fibrosis, colon fibrosis, or pancreatic fibrosis.

62. The method of claim 60, wherein the fibrosis is idiopathic pulmonary fibrosis.

63. The method of claim 60, wherein the fibrosis is the result of interstitial lung disease, viral infection, autoimmune disease, allergy, inhalation of occupational debris, dusts, fibers, fumes or vapors, inhalation of chemicals or molds, sepsis, adverse reaction to medications, aspirin overdose, hypersensitivity to environmental antigens, overexposure to chlorine, exposure to herbicides, fluorocarbons, radiation, chemotherapy, immune dysregulation, and/or treatment with one or more immune checkpoint inhibitors.

64. The method of claim 63, wherein the one or more checkpoint inhibitors are selected from an anti-programmed death receptor-1(PD1) molecule, an anti-programmed death ligand 1 (PD-L1) molecule, an anti-cytotoxic T-lymphocyte associated protein 4 (CTLA4) molecule, an anti-Lymphocyte-activation gene 3 (LAG3) molecule, an anti-T-cell immunoreceptor with Ig and ITIM domains (TIGIT) molecule, an anti-T-cell immunoglobulin and mucin domain-containing protein 3 (TIM-3) molecule, an anti-V-domain Ig suppressor of T cell activation (VISTA) molecule, an anti-B and T lymphocyte attenuator (BTLA) molecule, an anti-Sialic acid-binding Ig-like lectin 15 (Siglec-15) molecule, and an anti-CD96 molecule.

65. The method of claim 64, wherein the anti-PD1 molecule comprises an anti-PD1 antibody selected from a group consisting of pembrolizumab, nivolumab, cemiplimab, dostarlimab, spartalizumab, camrelizumab, sintilimab, tislelizumab, toripalimab, and retifanlimab.

66. The method of claim 64, wherein the anti-CTLA4 molecule comprises an anti-CTLA4 antibody selected from a group consisting of ipilimumab, tremelimumab, BMS-986249, quavonlimab, and AGEN1884.

67. The method of claim 64, wherein the anti-PD-L1 molecule comprises an anti-PD-L1 antibody selected from a group consisting of atezolizumab, avelumab, and durvalumab.

68. The method of claim 64, wherein the treatment with one or more checkpoint inhibitors comprises treatment with two checkpoint inhibitors.

69. The method of claim 68, wherein the two checkpoint inhibitors comprise an anti-PD1 antibody and an anti-CTLA4 antibody.

70. The method of claim 63, wherein the treatment with one or more checkpoint inhibitors comprises a cell therapy comprising CAT-T cells expressing one or more checkpoint inhibitors.

71. The method of claim 63, wherein the treatment with one or more checkpoint inhibitors comprises a cell therapy comprising allogeneic T cells expressing one or more checkpoint inhibitors.

72. The method of claim 63, wherein the treatment with one or more checkpoint inhibitors comprises a gene therapy comprising viral vectors expressing one or more checkpoint inhibitors.

73. The method of claim 63, wherein the treatment with one or more checkpoint inhibitors comprises one or more checkpoint inhibitors conjugated to a therapeutic moiety.

74. The method of claim 73, wherein the therapeutic moiety comprises a cytotoxic agent, a therapeutic agent, a radioisotope, an ultrasound sensitizer, or an exosome secretion inhibitor.

75. The method of claim 74, wherein the cytotoxic agent comprises taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof.

76. The method of claim 74, wherein the therapeutic agent comprises an antimetabolite, an alkylating agent, an anthracycline, an antibiotic, and an anti-mitotic agent.

77. The method of claim 76, wherein the antimetabolite comprises methotrexate, 6-mercaptopurine, 6-thioguanine, cytarabine, 5-fluorouracil dacarbazine.

78. The method of claim 76, wherein the alkylating agent comprises mechlorethamine, thioepa chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin.

79. The method of claim 76, wherein the anthracycline comprises daunorubicin and doxorubicin.

80. The method of claim 76, wherein the antibiotic comprises dactinomycin, bleomycin, mithramycin, and anthramycin (AMC).

81. The method of claim 76, wherein the anti-mitotic agent vincristine and vinblastine.

82. The method of claim 74, wherein the radioisotope is radioactive iodine.

83. The method of claim 74, wherein the ultrasound sensitizer comprises porphyrins, porphyrin isomers and expanded porphyrins.

84. The method of claim 74, wherein the exosome secretion inhibitor comprises Manumycin A, GW4869, cannabidiol and endothelin receptor antagonists.

85. The method of claim 60, wherein the treatment results in a reduction in total immune infiltrating cells in the bronchoalveolar space.

86. The method of claim 85, wherein the treatment results in a reduction in the ratio of lymphocytes relative to the total bronchoalveolar cells.

87. The method of claim 86, wherein the treatment results in a reduction in the ratio of neutrophils relative to the total bronchoalveolar cells.

88. The method of claim 60, wherein the treatment results in a reduction in total collagen.

89. The method of claim 60, wherein the treatment results in a reduction in fibronectin.

90. The method of claim 60, wherein the treatment results in a reduction in one or more molecular pro-fibrotic mediators.

91. The method of claim 90, wherein the molecular pro-fibrotic mediator is selected from transforming growth factor beta (TGF-β), connective tissue growth factor (CTGF), platelet-derived growth factor (PDGF), fibroblast growth factor (FGF), endothelin-1 (ET-1), IL-4, IL-5, IL-13, IL-21, MCP-1, MIP-1β, angiogenic factors (VEGF), TNF-α, peroxisome proliferator-activated receptors (PPARs), acute phase proteins (SAP), caspases, Angiotensin II, and endothelin (ET).

92. The method of any of claims 60 to 91, wherein the anti-alarmin binding molecule is administered as a single dose.

93. The method of any of claims 60 to 91, wherein the anti-alarmin binding molecule is administered as multiple doses.

94. A method of preventing or treating a type 2 inflammatory disease in a subject in need thereof comprising administering an anti-alarmin binding molecule to the subject.

95. The method of claim 94, wherein the type 2 inflammatory disease comprises asthma, viral exacerbations of allergic asthma, chronic rhinosinusitis with nasal polyps, allergic bronchopulmonary aspergillosis, atopic dermatitis, eosinophilic esophagitis, eosinophilic gastritis, eosinophilic gastroenteritis, colitis, allergic conjunctivitis, eosinophilia and food allergies.

96. The method of claim 94, wherein the type 2 inflammatory disease is a viral-induced type 2 inflammatory disease.

97. The method of claim 94, wherein the viral-induced type 2 inflammatory disease is selected from a group consisting of asthma, chronic obstructive pulmonary disease (COPD), eosinophilic esophagitis (EoE), chronic rhinosinusitis with nasal polyps (CRSwNP), and viral encephalitis, acute respiratory syndrome (SARS), Middle East respiratory syndrome (MERS), and COVID-19.

98. The method of claim 94, wherein the type 2 inflammatory disease is a type 2 inflammatory disease associated with allergic exacerbations.

99. The method of claim 98, wherein the type 2 inflammatory disease associated with allergic exacerbations is selected from a group consisting of asthma, allergic rhinitis, and atopic dermatitis.

100. The method of claim 94, wherein the type 2 inflammatory disease is a type 2 inflammatory disease associated with environmental exacerbations.

101. The method of claim 100, wherein the type 2 inflammatory disease associated with environmental exacerbations is selected from a group consisting of asthma, allergic rhinitis, and atopic dermatitis.

102. The method of claim 94, wherein the type 2 inflammatory disease is a type 2 inflammatory disease associated with drug-induced exacerbations.

103. The method of claim 102, wherein the drug is selected from non-steroidal anti-inflammatory drugs (NSAIDs), beta-blockers, ACE inhibitors, aspirin and one or more checkpoint inhibitors.

104. The method of claim 103, wherein the one or more checkpoint inhibitors are selected from an anti-programmed death receptor-1(PD1) molecule, an anti-programmed death ligand 1 (PD-L1) molecule, an anti-cytotoxic T-lymphocyte associated protein 4 (CTLA4) molecule, an anti-Lymphocyte-activation gene 3 (LAG3) molecule, an anti-T-cell immunoreceptor with Ig and ITIM domains (TIGIT) molecule, an anti-T-cell immunoglobulin and mucin domain-containing protein 3 (TIM-3) molecule, an anti-V-domain Ig suppressor of T cell activation (VISTA) molecule, an anti-B and T lymphocyte attenuator (BTLA) molecule, an anti-Sialic acid-binding Ig-like lectin 15 (Siglec-15) molecule, and an anti-CD96 molecule.

105. The method of claim 104, wherein the anti-PD1 molecule comprises an anti-PD1 antibody selected from a group consisting of pembrolizumab, nivolumab, cemiplimab, dostarlimab, spartalizumab, camrelizumab, sintilimab, tislelizumab, toripalimab, and retifanlimab.

106. The method of claim 104, wherein the anti-CTLA4 molecule comprises an anti-CTLA4 antibody selected from a group consisting of ipilimumab, tremelimumab, BMS-986249, quavonlimab, and AGEN1884.

107. The method of claim 104, wherein the anti-PD-L1 molecule comprises an anti-PD-L1 antibody selected from a group consisting of atezolizumab, avelumab, and durvalumab.

108. The method of claim 103, wherein the treatment with one or more checkpoint inhibitors comprises treatment with two checkpoint inhibitors.

109. The method of claim 108, wherein the two checkpoint inhibitors comprise an anti-PD1 antibody and an anti-CTLA4 antibody.

110. The method of claim 103, wherein the treatment with one or more checkpoint inhibitors comprises a cell therapy comprising CAT-T cells expressing one or more checkpoint inhibitors.

111. The method of claim 103, wherein the treatment with one or more checkpoint inhibitors comprises a cell therapy comprising allogeneic T cells expressing one or more checkpoint inhibitors.

112. The method of claim 103, wherein the treatment with one or more checkpoint inhibitors comprises a gene therapy comprising viral vectors expressing one or more checkpoint inhibitors.

113. The method of claim 103, wherein the treatment with one or more checkpoint inhibitors comprises one or more checkpoint inhibitors conjugated to a therapeutic moiety.

114. The method of claim 113, wherein the therapeutic moiety comprises a cytotoxic agent, a therapeutic agent, a radioisotope, an ultrasound sensitizer, or an exosome secretion inhibitor.

115. The method of claim 114, wherein the cytotoxic agent comprises taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs or homologs thereof.

116. The method of claim 114, wherein the therapeutic agent comprises an antimetabolite, an alkylating agent, an anthracycline, an antibiotic, and an anti-mitotic agent.

117. The method of claim 116, wherein the antimetabolite comprises methotrexate, 6-mercaptopurine, 6-thioguanine, cytarabine, 5-fluorouracil dacarbazine.

118. The method of claim 116, wherein the alkylating agent comprises mechlorethamine, thioepa chlorambucil, melphalan, carmustine (BSNU), lomustine (CCNU), cyclothosphamide, busulfan, dibromomannitol, streptozotocin, mitomycin C, and cis-dichlorodiamine platinum (II) (DDP) cisplatin.

119. The method of claim 116, wherein the anthracycline comprises daunorubicin and doxorubicin.

120. The method of claim 116, wherein the antibiotic comprises dactinomycin, bleomycin, mithramycin, and anthramycin (AMC).

121. The method of claim 116, wherein the anti-mitotic agent vincristine and vinblastine.

122. The method of claim 114, wherein the radioisotope is radioactive iodine.

123. The method of claim 114, wherein the ultrasound sensitizer comprises porphyrins, porphyrin isomers and expanded porphyrins.

124. The method of claim 114, wherein the exosome secretion inhibitor comprises Manumycin A, GW4869, cannabidiol and endothelin receptor antagonists.

125. The method of any of claims 94 to 124, wherein the treatment results in a reduction in type 2 cytokine expression.

126. The method of claim 125, wherein the type 2 cytokine comprises IL13, IL4 and IL5.

127. The method of any of claims 94 to 124, wherein the treatment results in a reduction in cotaxin and eosinophils.

128. The method of any of claim 94 to 124, wherein the anti-alarmin binding molecule is administered as a single dose.

129. The method of any of claim 94 to 124, wherein the anti-alarmin binding molecule is administered as multiple doses.

130. The method of any one of claims 1 to 129, wherein the anti-alarmin binding molecule is selected from a group consisting of an anti-IL-25 antibody, an anti-IL-33 antibody, and an anti-TSLP antibody.

131. The method of claim 130, wherein the anti-IL-33 antibody is selected from tozorakimab and itepekimab.

132. The method of claim 130, wherein the anti-TSLP antibody is Tezepelumab.

133. The method of claim 130, wherein the anti-IL-25 antibody comprises a heavy chain variable domain comprising a HCDR1 of SEQ ID NO: 1, a HCDR2 of SEQ ID NO: 2, and a HCDR3 of SEQ ID NO: 3.

134. The method of claim 133, wherein the heavy chain variable domain comprises the sequence of SEQ ID NO: 4.

135. The method of claim 130, wherein the anti-IL-25 antibody comprises a heavy chain variable domain comprising a HCDR1 of SEQ ID NO: 9, a HCDR2 of SEQ ID NO: 10, and a HCDR3 of SEQ ID NO: 11.

136. The method of claim 135, wherein the heavy chain variable domain comprises the sequence of SEQ ID NO: 12.

137. The method of any one of claims 133-136, wherein the anti-IL25 binding molecule comprises a light chain variable domain comprising a LCDR1 of SEQ ID NO: 5, a LCDR2 of SEQ ID NO: 6, and a LCDR3 of SEQ ID NO: 7.

138. The method of claim 137, wherein the light chain variable domain comprises the sequence of SEQ ID NO: 8 or SEQ ID NO: 13.

139. The method of claim 130, wherein the anti-IL-25 antibody comprises a heavy chain variable domain comprising a HCDR1 of SEQ ID NO: 1, a HCDR2 of SEQ ID NO: 2, and a HCDR3 of SEQ ID NO: 3, and a light chain variable domain comprising a LCDR1 of SEQ ID NO: 5, a LCDR2 of SEQ ID NO: 6, and a LCDR3 of SEQ ID NO: 7.

140. The method of claim 139, wherein the heavy chain variable domain comprises the sequence of SEQ ID NO: 4 and the light chain variable domain comprises the sequence of SEQ ID NO: 8.

141. The method of claim 130, wherein the anti-IL-25 antibody comprises a heavy chain variable domain comprising a HCDR1 of SEQ ID NO: 9, a HCDR2 of SEQ ID NO: 10, and a HCDR3 of SEQ ID NO: 11, and a light chain variable domain comprising a LCDR1 of SEQ ID NO: 5, a LCDR2 of SEQ ID NO: 6, and a LCDR3 of SEQ ID NO: 7.

142. The method of claim 139, wherein the heavy chain variable domain comprises the sequence of SEQ ID NO: 12 and the light chain variable domain comprises the sequence of SEQ ID NO: 13.

143. The method of any of claims 133-142, wherein the anti-IL-25 antibody is administered intraperitoneally.

144. The method of any of claims 133-142, wherein the anti-IL-25 antibody is administered subcutaneously.

145. The method of any of claims 133-142, wherein the anti-IL-25 antibody is administered intravenously.

146. The method of any of claims 133-142, wherein the therapeutically effective dose of the anti-IL25 binding molecule is 5 mg/kg, 10 mg/kg, 20 mg/kg, 30 mg/kg, 40 mg/kg, or 50 mg/kg.
